(19) United States
(12) Patent Application Publication
(10) Pub. No.: US 2010/0151572 A1
(43) Pub. Date: Jun. 17, 2010

(54) ANTI-TUMORAL COMPOSITIONS METHODS

(76) Inventors: Eric J. Sorscher, Birmingham, AL (US); William B. Parker, Birmingham, AL (US); Jeong S. Hong, Birmingham, AL (US)

Correspondence Address: GIFFORD, KRASS, SPRINKLE, ANDERSON &;CITKOWSKI, P.C., P.O. BOX 7021, TROY, MI, 48007-7021, US

(21) Appl. No.: 11/914,563

(22) PCT Filed: May 16, 2006
(86) PCT No.: PCT/US2006/018844

§ 371 Date: May 19, 2008

Related U.S. Application Data
(60) Provisional application No. 60/681,305, filed on May 16, 2005.

(51) Int. Cl. C12N 5/02; C12N 15/63

(52) U.S. Cl. 435/375; 435/320.1

(57) ABSTRACT

Described are methods and compositions for inhibiting undesired cells by expression of one or more exogenous enzymes in the cells and administration of a prodrug which is a substrate for at least one of the enzymes to produce a cytotoxic compound. Inventive methods and compositions are active to inhibit cells expressing the exogenous enzymes as well as bystander cells. Tumor cells are a particular target for inhibition using methods and compositions detailed according to the present invention. Provided are methods and compositions for improved anti-tumoral effects by overexpression of adenine phosphoribosyltransferase (APRT) to produce cytotoxins which inhibit the cells overexpressing the APRT as well as bystander cells. Overexpression of APRT in conjunction with expression of E. coli PNP and administration of a substrate for E. coli PNP provides particularly effective anti-tumoral effects.

### REFERENCE TO RELATED APPLICATION

This application claims priority of U.S. Provisional Patent Application Ser. No. 60/681,305, filed May 16, 2005, the entire content of which is incorporated herein by reference.

### GRANT REFERENCE

Research carried out in connection with this invention was supported in part by NIH grant U19CA67763. Accordingly, the United States government may have certain rights in the invention.

### FIELD OF THE INVENTION

The present invention generally relates to anti-tumoral compositions and methods.

### BACKGROUND OF THE INVENTION

Most conventional chemotherapeutic drugs derive anti-tumor specificity from the ability to kill dividing, as opposed to non-dividing, cells. Many chemotherapies are suitable for systemic administration specifically because they are most toxic to cells that are dividing. This leads to an acceptable level of damage in other, rapidly proliferating, tissues and cells such as the bone marrow, intestinal tract and hair follicles, among others. However, many refractory tumors are refractory precisely because they have a very low growth fraction; i.e. a relatively small percentage of tumor cells are dividing at any particular point in time. For example, it has been estimated in hormone refractory prostate cancer that approximately 5% of the cells are dividing, and that the low growth fraction explains the poor response to therapy as described in Dionne, C. A., et al., 1998, Clin. Cancer Res. 4(8), 1887-98; Sai, S., et al., 1991, Hinyokika Kiyo-Acta Urologica Japonica. 37, 881-6; and Sadi, M. V. and Barrack, E. R., 1991, Cancer 67, 3065-3071. In refractory colon, breast, glioma, melanoma, and non-small cell lung cancers, the growth fractions are estimated at 4%, 4%, 10-30%, 10-40% and approximately 40% respectively, as detailed in Tay, D. L., et al., 1991, J. Clin. Invest. 87: 519-527; Giangaspero, F., et al., 1987, Acta Neuropathologica. 74, 179-82; Pierard, G. E., and Pierd-Franchimont, C., 1997, Euro. J. Cancer 33: 1888-1892; Crafts, D. C., et al., 1977, Bulletin du Cancer 64:115-24; Fontanini, G., et al., 1992, Am. J. Pathology 141, 1285-1290; and Vescio, R. A., et al., 1990, Cancer Res. 50: 6095-6099. Even if 75% of the cells in an otherwise refractory tumor were proliferating during a cumulative exposure to chemotherapy, and even if all these cells were completely eradicated by treatment, the tumor would remain at two doubling times away from regaining pretreatment dimensions. As a consequence, it may be unrealistic to expect drugs that kill mainly dividing cells to cure large tumors of these types. This explanation is generally acknowledged as a reason for the failure of conventional chemotherapy against many tumors.

Thus, there is a continuing need for improved compositions and methods for inhibiting undesired cells, such as tumor cells.

### SUMMARY OF THE INVENTION

A method of inhibiting a mammalian cell is provided according to the present invention which includes introducing an expression vector including a nucleotide sequence encoding an adenine phosphoribosyltransferase (APRT or APRTase) into the mammalian cell and contacting the mammalian cell with an effective amount of a substrate for the adenine phosphoribosyltransferase. Activation of this substrate by the adenine phosphoribosyltransferase yields a compound which is toxic to the mammalian cell and which inhibits the cell. In addition, bystander cells are inhibited according to an inventive method.

The encoded adenine phosphoribosyltransferase is preferably a mammalian adenine phosphoribosyltransferase, such as a human adenine phosphoribosyltransferase.

In one embodiment, a nucleic acid sequence encoding human adenine phosphoribosyltransferase encodes amino acids 1-180 of Seq ID No 1.

An expression vector including a nucleic acid encoding an adenine phosphoribosyltransferase may be any of various types of expression vector. General expression vector types include plasmids and viruses.

Illustrative viral expression vectors include an adenovirus, a herpes virus, an adeno-associated virus and a lentivirus.

A substrate for adenine phosphoribosyltransferase is a purine analog, illustratively including 6-methylpurine and 2-fluoroadenine.

A mammalian cell inhibited according to a method of the present invention may be a tumor cell in a subject. In further embodiments, an inventive method inhibits a mammalian cell in a subject which is abnormal or which is contributing to a disease or other pathological process.

In a further embodiment, a method of inhibiting a mammalian cell includes introducing a first expression vector including a nucleotide sequence encoding a prokaryotic purine nucleoside phosphorylase (PNP) and a second expression vector comprising a nucleotide sequence encoding an adenine phosphoribosyltransferase into the mammalian cell. The cell is contacted with an effective amount of a prodrug which is a substrate for the purine nucleoside phosphorylase. Cleavage of the prodrug by the purine nucleoside phosphorylase yields a substrate for the adenine phosphoribosyltransferase. Subsequent activation of the adenine phosphoribosyltransferase substrate by enzymatic action of APRT yields a compound toxic to the mammalian cell, thereby inhibiting the cell. In addition, bystander cells are inhibited according to an inventive method.

In a preferred embodiment, the prokaryotic purine nucleoside phosphorylase is an E. coli purine nucleoside phosphorylase.

The expression vectors including the nucleotide sequence encoding a prokaryotic purine nucleoside phosphorylase and the nucleotide sequence encoding an adenine phosphoribosyltransferase may be the same type of vector or different types of vector.

Each vector may independently be a plasmid or a virus, for instance. In a preferred embodiment, the expression vectors encoding the purine nucleoside phosphorylase and the adenine phosphoribosyltransferase are both plasmids or both viruses. A single expression vector may contain a nucleic acid encoding both the purine nucleoside phosphorylase and the adenine phosphoribosyltransferase. For example, a bicistronic nucleic acid is optionally included in an expression vector. A bicistronic nucleic acid for expression of two proteins may include an internal ribosome entry site (IRES), permitting translation of two open reading frames from one mRNA.

The substrate for the purine nucleoside phosphorylase includes a purine nucleoside analog which is non-toxic to cells and which is capable of being cleaved by a purine nucleoside phosphorylase to yield a substrate for adenine phosphoribosyltransferase.

A composition is provided which includes a bicistronic expression construct including a first nucleic acid encoding a prokaryotic purine nucleoside phosphorylase and a second nucleic acid encoding a mammalian adenine phosphoribosyltransferase, the first and second nucleic acids both operably linked to a promoter. Such a composition is useful in methods to express the encoded proteins and particularly in methods for inhibiting a cell according to the present invention.

Preferred are embodiments in which the bicistronic expression construct further includes an internal ribosome entry site disposed between the first nucleic acid encoding a prokaryotic purine nucleoside phosphorylase and the second nucleic acid encoding a mammalian adenine phosphoribosyltransferase.

In particular embodiments, a first nucleic acid encoding a prokaryotic purine nucleoside phosphorylase encodes an E. coli purine nucleoside phosphorylase. This first nucleic acid encoding an E. coli purine nucleoside phosphorylase preferably encodes a protein which is at least 90% identical to a E. coli purine nucleoside phosphorylase of SEQ ID No. 3. In addition, the first nucleic acid encoding a prokaryotic purine nucleoside phosphorylase is generally at least 80% identical to a E. coli purine nucleoside phosphorylase encoding portion of a nucleic acid of SEQ ID No. 4.

The second nucleic acid encoding a mammalian adenine phosphoribosyltransferase optionally and preferably encodes a human adenine phosphoribosyltransferase. The second nucleic acid encoding a human adenine phosphoribosyltransferase preferably encodes a protein which is at least 90% identical to a human adenine phosphoribosyltransferase of SEQ ID No. 1. Further optionally and preferably, the second nucleic acid encoding a human adenine phosphoribosyltransferase is at least 80% identical to a human adenine phosphoribosyltransferase encoding portion of a nucleic acid of SEQ ID No. 2.

An expression vector is provided according to the present invention which includes a nucleic acid sequence encoding a mammalian adenine phosphoribosyltransferase. In preferred embodiments, particularly, where a human cell is to be inhibited, a human adenine phosphoribosyltransferase is encoded. A human adenine phosphoribosyltransferase nucleic acid encoding a human adenine phosphoribosyltransferase is preferably at least 80% identical to a human adenine phosphoribosyltransferase encoding portion of a nucleic acid of SEQ ID No. 2. In further embodiments, the nucleic acid encoding a human adenine phosphoribosyltransferase encodes a protein which is at least 90% identical to a human adenine phosphoribosyltransferase of SEQ ID No. 1.

A pharmaceutical composition for inhibiting a cell is provided which includes an expression vector including a nucleotide sequence encoding an adenine phosphoribosyltransferase. A pharmaceutically acceptable carrier is also included in such a pharmaceutical composition.

Optionally, certain embodiments of a pharmaceutical composition according to the present invention include an expression vector which includes a nucleotide sequence encoding a prokaryotic purine nucleoside phosphorylase.

A further embodiment of such a composition is provided in which the expression vector including a nucleotide sequence encoding an adenine phosphoribosyltransferase and the expression vector including a nucleotide sequence encoding a prokaryotic purine nucleoside phosphorylase are the same vector, the nucleotide sequence encoding an adenine phosphoribosyltransferase and the nucleotide sequence encoding a prokaryotic purine nucleoside phosphorylase operably connected to a regulatory element in a bicistronic nucleic acid.

Antibodies recognizing a prokaryotic purine nucleoside phosphorylase are provided according to the present invention. Such antibodies include both monoclonal and polyclonal antibodies capable of specifically detecting E. coli PNP by immunofluorescence detection, immunoprecipitation, immunoblotting and/or ELISA.

### BRIEF DESCRIPTION OF THE DRAWINGS

FIG. 1 is a schematic illustration of the role of APRTase in tumor cell killing in the context of recombinant E. coli PNP and APRT delivery and administration of particular prodrugs.

FIG. 2 illustrates glycosidic cleavage of nucleoside prodrugs to purine bases.

FIG. 3 is an image of a fluorescently labeled nucleic acid on a gel illustrating a 543 by band which is a PCR product encoding full length human APRT.

FIG. 4 is a graphic representation of an expression vector according to the present invention containing a DNA sequence encoding human APRT.

FIG. 5 is an image of immunoprecipitated E. coli PNP detected using an antibody according to the present invention.

FIG. 6 is an image of E. coli PNP detected by Western blot using an antibody according to the present invention.

FIG. 7 is a graph illustrating generation of F-Ade from the active PNP substrate, F-dAdo.

FIG. 8 is an image showing in vitro bystander activity of MeP-dR in D54 human glioma cells expressing E. coli PNP.

FIG. 9 is a graph illustrating bystander killing by MeP-dR when E. coli PNP is expressed using a MuLV expression vector.

FIG. 10 is a graph illustrating effects of recombinant lentivirus expression of E. coli PNP on D54 glioma tumors in vivo with and without a prodrug.

FIG. 11 is a graph illustrating effects of recombinant lentivirus expression of E. coli PNP on D54 glioma tumors in vivo in which only 1% of the tumor cells are PNP expressing cells.

FIG. 12 is a graph illustrating a study in which 100% of cells express a lentivirus encoded transgene.

FIG. 13 is a graph illustrating dose dependence upon the amount of prodrug added.

FIG. 14 is a graph illustrating that tumors with lower proportions of PNP-expressing cells (10%, 5%, 2.5%), exhibit dose dependence upon intratumoral PNP activity.

FIG. 15 is a graph illustrating effects of control treatments to be compared with graphs in FIGS. 13 and 14.

FIG. 16 is a graph illustrating some effects of different schedules of prodrug dosing.

FIG. 17 is an image illustrating cell killing effects using an Ela deleted adenoviral vector encoding a transgene according to the present invention.

FIG. 18 is a graph illustrating anti-tumor effects of F-araAMP following delivery of an adenovirus expression vector according to the present invention.

### DETAILED DESCRIPTION OF THE INVENTION

Described are methods and compositions for inhibiting undesired cells by expression of one or more exogenous enzymes in the cells and administration of a prodrug which is a substrate for at least one of the enzymes to produce a cytotoxic compound. Inventive methods and compositions are active to inhibit cells expressing the exogenous enzymes as well as bystander cells. Bystander cells are cells other than those in which the exogenous enzymes are expressed.

The term “inhibiting a mammalian cell” in the context of a process according to the present invention refers to disruption of cellular processes, such as transcription, translation and ATP-dependent processes. Death of the mammalian cell results from inhibition.

Compositions and methods active against both dividing and non-dividing cells designed to inhibit tumors with a low growth fraction are provided according to the present invention. Specific strategies to kill low growth fraction tumors are required to eliminate the common cancers most refractory to conventional treatment.

In one embodiment, a method of inhibiting a mammalian cell is provided according to the present invention which includes introducing an expression vector including a nucleotide sequence encoding an adenine phosphoribosyltransferase (APRT or APRTase) into the mammalian cell and contacting the mammalian cell with an effective amount of a substrate for the adenine phosphoribosyltransferase. Activation of this substrate by the adenine phosphoribosyltransferase yields a compound which is toxic to the mammalian cell.

APRT is an enzyme which belongs to the purine/pyrimidine phosphoribosyltransferase family. Among other reactions, the enzyme catalyzes the formation of AMP and inorganic pyrophosphate from adenine and 5-phosphoribosyl-1-pyrophosphate. Purine bases are substrates for cellular APRTases, which convert the compounds to phosphorylated metabolites. Because the APRTase carries out the first step in intracellular adenine activation, this enzyme is expressed in most cells, as well as in malignant cell types and tissues. In the context of the present invention, APRT is active to convert a purine analog to a cytotoxic compound, particularly a cytotoxic nucleotide analog. Such cytotoxic nucleotide analogs are incorporated into cellular RNA, disrupting both RNA and protein synthesis. In addition, such compounds may inhibit ATP dependent processes in the cell. Cell death results from such disruptions, causing RNA degradation and release of the cytotoxic nucleotide analogs to be taken up by bystander cells. Examples of such purine analogs include MeP and 2-fluoroadenine.

In a further embodiment, a method of inhibiting a mammalian cell includes introducing a first expression vector including a nucleotide sequence encoding a prokaryotic purine nucleoside phosphorylase (PNP) and a second expression vector comprising a nucleotide sequence encoding an adenine phosphoribosyltransferase into the mammalian cell. The cell is contacted with an effective amount of a prodrug which is a substrate for the purine nucleoside phosphorylase. Cleavage of the prodrug by the purine nucleoside phosphorylase yields a substrate for the adenine phosphoribosyltransferase. Subsequent activation of the adenine phosphoribosyltransferase substrate by APRT enzymatic action yields a compound toxic to the mammalian cell, thereby inhibiting the cell.

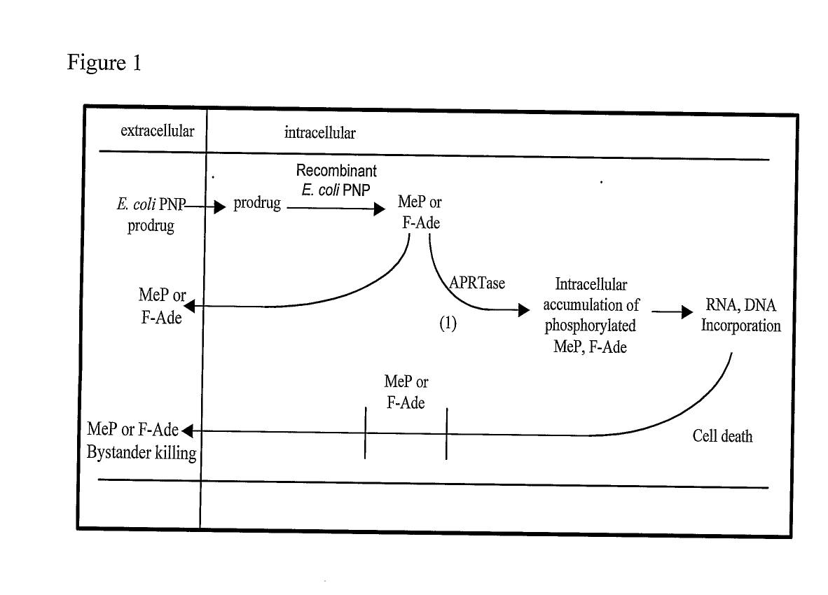

FIG. 1 illustrates the role of APRTase in tumor cell killing in the context of recombinant E. coli PNP delivery and administration of particular prodrugs. APRTase is rate limiting for conversion of the purine base analogs, MeP and F-Ade, produced by PNP cleavage of the prodrugs, to a phosphorylated form. Thus, increased APRTase may shift the equilibrium of the reaction shown at (1) towards intratumoral accumulation of the toxic bases.

APRTase represents the rate limiting step for PNP toxin activation in vitro as described in Parker, W. B., et al., 1998, Biochem. Pharmacol. 55:1673-1681. APRTase governs the pathway by which MeP and F-Ade become phosphorylated, trapped in tumor cells, and mediate anti-tumor effects, illustrated in FIG. 1. After tumor cells die, MeP and F-Ade are regenerated, released, and recycled to neighboring tumor cells to elicit further rounds of bystander killing.

Administration of a prokaryotic PNP and APRT allow for improved cell inhibitory effects compared to administration of either agent alone. In particular, as noted herein, APRT activity represents a rate limiting step in producing cytotoxins from administered prodrug substrates. Overexpression of APRT in conjunction with delivery of a prokaryotic PNP provides for increased toxin production, increased cell inhibition and increased bystander cell inhibition. In particular, in methods of treating tumors and inhibiting tumor cells, increased tumor regressions are provided with administration of both a prokaryotic PNP and APRT. Such methods allow shorter treatment times and better effects with large tumor masses.

Compounds produced by E. coli PNP cleavage of prodrugs, such as MeP and F-Ade, are substrates for APRT. The toxins liberated by E. coli PNP are activated by APRT to cytotoxic metabolites which are incorporated into cellular RNA, disrupting both RNA and protein synthesis. Cell death results over a period of days, causing RNA degradation and release of the toxins from nucleic acid pools into the extra-cellular space.

### Expression Vectors

An expression vector including a nucleic acid encoding an adenine phosphoribosyltransferase and/or prokaryotic PNP may be any of various types of expression vector. In the context of the present invention a suitable vector is adapted to express APRT and/or PNP in a mammalian cell. Such vectors include vectors derived from bacterial plasmids and from viruses such as adenoviruses; adeno-associated viruses; papovaviruses such as SV40; poxviruses; pseudorabies viruses; retroviruses such as lentiviruses; herpesviruses; and vaccinia viruses. An expression vector which is a virus may be replication-competent, conditionally replication-competent or replication defective. Various cloning and expression vectors are described in Sambrook et al., Molecular Cloning: A Laboratory Manual. 2nd. ed., Cold Spring Harbor Laboratory Press, Cold Spring Harbor, N.Y., (1989).

General expression vector types include plasmids and viruses containing one or more regulatory elements sufficient or desirable for expressing an encoded transgene.

The term “expression vector” as used herein refers to a recombinant DNA molecule containing a desired nucleic acid coding sequence encoding APRT and/or a prokaryotic PNP, and containing appropriate regulatory elements necessary or desirable for the expression of the operably linked coding sequence in a particular cell.

The term “regulatory element” as used herein refers to a nucleotide sequence which controls some aspect of the expression of nucleic acid sequences. Exemplary regulatory elements illustratively include an enhancer, an internal ribosome entry site (“IRES”), an origin of replication, a polyadenylation signal, a promoter, a transcription termination sequence, and an upstream regulatory domain, which contribute to the replication, transcription, post-transcriptional processing and/or translation of a coding sequence and/or encoded polypeptide in a cell.

The term “operably linked” as used herein refers to connection of two or more nucleic acid molecules, including a nucleic acid sequence to be transcribed to produce an mRNA encoding a desired peptide or protein and a regulatory element such as a promoter or an enhancer element, which allows transcription of the nucleic acid sequence to be transcribed.

The term “promoter,” as used herein, refers to a DNA sequence operably linked to a desired nucleic acid sequence encoding APRT and/or a prokaryotic PNP which is capable of controlling the transcription of the nucleic acid sequence. A promoter is generally positioned upstream of a desired nucleic acid sequence encoding APRT and/or a prokaryotic PNP to direct transcription, although a promoter may be positioned alternatively. A promoter may provide a site for specific binding of various factors involved in transcription, such as an RNA polymerase and/or other transcription factors.

A promoter included in an inventive expression vector may be a promoter naturally associated with APRT or PNP. Alternatively, a heterologous promoter may be used. Promoters drive constitutive expression, cell or tissue specific expression and/or regulated or inducible expression. Exemplary constitutive promoters include viral promoters such as CMV, SV40, and RSV promoters. Exemplary constitutive mammalian promoters include the beta-actin promoter, the ubiquitin-1 promoter and the glyceraldehyde dehydrogenase promoter. Additional promoters are known in the art and some specific promoters are described in examples herein. Further suitable regulatory elements include, the egr-1 promoter, the EF-1alpha promoter, the WPRE regulatory element and hypoxia responsive elements.

A substrate for adenine phosphoribosyltransferase administered according to the present invention is a purine analog which is converted to a cytotoxic nucleotide analog by adenine phosphoribosyltransferase. Such purine analogs illustratively include 6-methylpurine and 2-fluoroadenine.

A mammalian cell inhibited according to a method of the present invention may be a tumor cell.

In further embodiments, it is desirable to inhibit a mammalian cell which is inhibited is abnormal or which is contributing to a disease or other pathological process.

For example, a cell infected with a microbe may be inhibited according to an inventive process in order to eliminate the cell and microbe. Such microbes include bacteria, viruses and protozoa. In a further example, cells contributing to inflammatory processes causing pain or degeneration, as in rheumatoid arthritis, may be inhibited.

A vector administered according to methods of the present invention may be targeted to particular cells. Targeting may be achieved by association of the vector with a targeting moiety. For example, a targeting moiety may be a receptor ligand, an antibody, a lectin or other binding partner specific for a complementary receptor on a target cell, such as a tumor cell.

A vector may be administered in conjunction with a transfection or transduction enhancer in embodiments of the invention.

For example, a gene delivery compound may be used in conjunction with virus vectors. Gene delivery compounds are active to stimulate uptake of a virus into a cell. Such compounds are described in U.S. Patent Publication 20040204375 and U.S. patent application Ser. No. 10/520,377.

Additional adjunctive compounds for stimulating uptake and/or transgene expression of vectors encoding PNP and/or APRT may be used. Such adjunctive compounds illustratively include liposomal formulations, alginate formulations, or poloxamer installation such as described in Toyoda K, et al., 2001, J Cereb Blood Flow Metab., 21(9):1125-31; Wang Y, et al., 2005, Cancer Res., 65(17):7541-5; Varga C M, et al., 2005, Gene Ther., 12(13):1023-32; Clark P R, et al., 1999, Cancer Gene Ther., 6(5):437-46; Fasbender A, et al., 1998, J Clin Invest., 102(1): 184-93; and Fasbender A, et al., 1997, J Biol Chem., 272(10):6479-89.

The expression vectors including the nucleotide sequence encoding a prokaryotic purine nucleoside phosphorylase and the nucleotide sequence encoding an adenine phosphoribosyltransferase may be the same type of vector or different types of vector. Each vector may independently be a plasmid or a virus, for instance. In a preferred embodiment, the expression vectors encoding the purine nucleoside phosphorylase and the adenine phosphoribosyltransferase are both plasmids or both viruses.

A composition provided according to one embodiment of the present invention includes a single expression vector containing a nucleic acid encoding both a prokaryotic purine nucleoside phosphorylase and an adenine phosphoribosyltransferase. For example, a bicistronic nucleic acid is included in an expression vector. A bicistronic nucleic acid for expression of two proteins may include an internal ribosome entry site (IRES), permitting translation of two open reading frames from one mRNA. IRES are exemplified by the encephaomyocarditis virus IRES described in Jang et al., J. Virol., 1988, 62, 2636-2643.

A nucleic acid sequence encoding an APRT is preferably a mammalian APRT. Exemplary nucleic acid sequences encoding mammalian APRTases include those detailed in Sikela J M, et al., Gene, 1983, 22(2-3):219-28; Stambrook P J, et al., Somat Cell Mol Genet., 1984, 10(4):359-67; Lowy I, et al., Cell, 1980, 22(3):817-23; Wilson, J. M., et al., J. Biol. Chem., 1986, 261:13677-13683; and Murray A M, et al., Gene, 1984, 31(1-3):233-40.

A human APRT cDNA is isolated as described in further detail in examples included herein. Cloning and expression vectors are provided according to embodiments of the present invention which contain a nucleic acid sequence encoding a human APRT of SEQ ID No. 1.

A nucleic acid sequence encoding a prokaryotic PNP encodes an E. coli PNP in a preferred embodiment. Nucleic acid sequences encoding E. coli PNP as well as cloning and expression vectors containing such sequences are described in detail in examples included herein, in U.S. Pat. Nos. 5,552,311; 6,017,896; 6,491,905; 6,958,318 and 7,037,718; and in U.S. Patent Application Publication Nos. 2005/0214901; 20040204375; 2003/0228576; 2003/0134819; and 2003/0077268. In addition, mutant E. coli PNPs are detailed in U.S. Pat. No. 7,037,718 which are suitable for use in methods and compositions according to the present invention. A wild-type E. coli protein is detailed in the present specification in SEQ ID No. 3.

An isolated nucleic acid sequence encoding human APRT or E. coli PNP may be identical to the coding portion of sequences shown in SEQ ID No. 2 or SEQ ID No. 4, respectively. Alternatively, a different isolated nucleic acid encoding a protein having activity substantially similar to human APRT or E. coli PNP, as shown in SEQ ID No. 1 or SEQ ID No. 3, respectively, may be used owing to the redundancy or degeneracy of the genetic code. In general, an isolated nucleic acid sequence encoding human APRT or E. coli PNP is at least 80%, 85% or 90% identical to the nucleic acid sequences of SEQ ID No. 2 or SEQ ID No. 4. In further embodiments, an isolated nucleic acid sequence encoding human APRT or E. coli PNP is at least 91%, 92%, 93%, 94%, 95%, 96%, 97%, 98%, or 99% identical to the nucleic acid sequences of SEQ ID No. 2 or SEQ ID No. 4.

In some embodiments, nucleic acids encoding APRT and/or PNPs include coding sequences for conservative amino acid substitutions which have little or no effect on enzyme activity compared to the wild-type proteins. The enzyme activity of mutant APRT and PNPs may be assessed by functional assays, such as those described herein. A conservatively modified APRT or prokaryotic PNP is one which includes a substitution of an amino acid present in the wild-type protein with a chemically similar amino acid. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine tyrosine and tryptophan; polar amino acids include aspartate, glutamate, histidine, lysine, asparagine, glutamine, arginine, serine, threonine and tyrosine; hydrophobic amino acids include alanine, cysteine, phenylalanine, glycine, isoleucine, leucine, methionine, proline, threonine, valine, tyrosine and tryptophan; and hydrophilic amino acids include. Amino acids may also be described in terms of relative size, alanine, cysteine, aspartate, glycine, asparagine, proline, threonine, serine, valine, all typically considered to be small.

In general, where an encoded protein includes a conservative substitution, an isolated nucleic acid sequence encoding human APRT or E. coli PNP encodes a protein which is at least 95%, 96%, 97%, 98%, or 99% identical to the nucleic acid sequences of SEQ ID No. 1 or SEQ ID No. 3.

In additional embodiments, mutant APRT and PNPs may be generated which have substantially similar or better enzyme activity compared to the wild-type proteins. The enzyme activity of mutant APRT and PNPs may be assessed by functional assays, such as those described herein. In general, an isolated nucleic acid sequence encoding mutant human APRT or E. coli PNP encodes a protein which is at least 95%, 96%, 97%, 98%, or 99% identical to the nucleic acid sequences of SEQ ID No. 1 or SEQ ID No. 3. Certain specific mutants of E. coli PNP which may be used include M65V and A157V, among others, as described in detail in U.S. Pat. No. 7,037,718.

The term “isolated” as used herein to refer to a nucleic acid or amino acid sequence is intended to indicate that the nucleic acid or amino acid sequence has been removed from the original environment in which it naturally occurs and separated from other nucleic acids present in the natural source of the molecule.

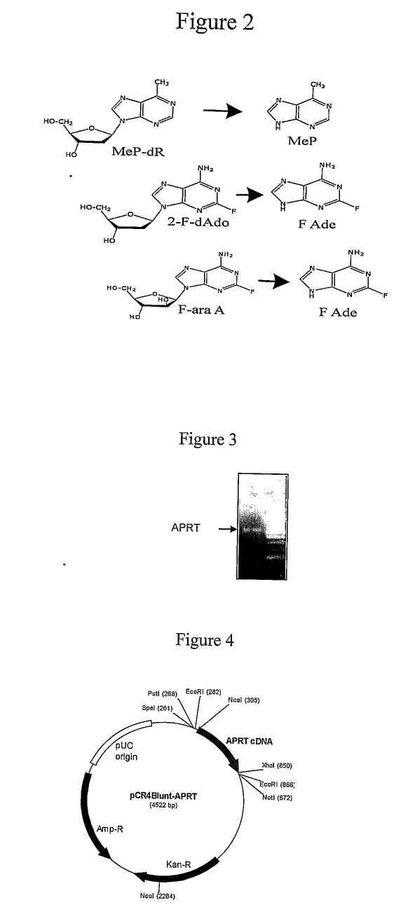

Prokaryotic PNP mediates the glycosidic cleavage of nucleoside prodrugs, to highly toxic purine bases. FIG. 2 illustrates specific examples of such cleavage, showing conversion of MeP-dR to MeP and 2-F-dAdo to F-Ade. Also shown is cleavage of F-araA, the bioavailable form of the clinically approved chemotherapeutic fludarabine monophosphate, to F-Ade.

Table 1 describes the kinetic constants underlying these enzymatic reactions with E. coli PNP.

TABLE 1
Kinetic Constants for MeP-dR, F-dAdo and
F-araA, with E. coli PNP

| Prodrug | Kₘ (μM) | Vₘₐₓ (nmol/mg/h) |
|---|---|---|
| MeP-dR | 125 | 1,460,000 |
| 2-F-dAdo | 22 | 425,000 |
| F-araA | 960 | 5,900 |

While reversible phosphorolysis of (2′-deoxy) purine ribonucleosides to free base and (2′deoxy) ribose-1-phosphate is a shared property of prokaryotic and eukaryotic PNPs, the mammalian enzymes differ fundamentally in sequence, structure and function from their bacterial counterparts (Mao, C., et al., 1997, Structure 5 (10), 1373-1383; Ealick, S. E., et al., 1990, J. Biol. Chem. 265(3), 1812-20; Zimmerman, T. P., et al., 1971, Can. J. Biochem. 49, 1050-1054; and Jensen, K. F., and Nygaard, P., 1975, Eur. J. Biochem. 51, 253-265.). Prokaryotic PNP's are hexameric (believed to act as a set of functional dimers), while the mammalian enzymes exist as trimers. Moreover, mammalian enzymes accept only guanosine and inosine as substrates, whereas bacterial enzymes such as E. coli PNP also accept adenosine, and are permissive for several ribose modifications. The structural and functional dichotomy is also reflected in the activity of enzymatic blockers. Formycin A, for example, abrogates E. coli PNP activity with a kᵢ=5 micromolar, but is inactive against the human enzyme.

The substrate for the purine nucleoside phosphorylase includes a purine nucleoside analog which is non-toxic to cells and which is capable of being cleaved by a purine nucleoside phosphorylase to yield a substrate for adenine phosphoribosyltransferase. Examples of such purine nucleoside analogs include 9-(2-deoxy-beta-D-ribofuranosyl]-6-methylpurine; 9-(beta-D-ribofuranosyl)-2-amino-6-chloro-1-deazapurine; 7-(beta-D-ribofuranosyl)-3-deazaguanine; 9-(beta-D-arabinofuranosyl)-2-fluoroadenine; 2-fluoro-2′-deoxyadenosine; 9-(5-deoxy-beta-D-ribofuranosyl)-6-methylpurine; 2-fluoro-5′-deoxyadenosine 2-chloro-2′-deoxyadenosine; 5′-amino-5′-deoxy-2-fluoroadenosine; 9-(5-amino-5-deoxy-beta-D-ribofuranosyl)-6-methylpurine; 9-(alpha-D-ribofuranosyl)-2-fluoroadenine; 9-(2,3-dideoxy-beta-D-ribofuranosyl)-6-methylpurine; 2′,3′-dideoxy-2-fluoroadenosine; 9-(3-deoxy-beta-D-ribofuranosyl]-6-methylpurine; 2-fluoro-3′-deoxyadenosine. Combinations of such substrates include mixtures of two or more substrates.

Additional substrates suitable for use with wild type and mutant E. coli PNPs are disclosed in U.S. Pat. No. 7,037,718.

### Multi-Modality Methods and Compositions

Methods and compositions are provided for multi-modality approaches to inhibition of cells, and particularly for inhibition of tumors. For example, administration of radiation or conventional chemotherapy is a contemplated embodiment for enhancement of anti-tumor methods and compositions including APRT and/or PNP according to the present invention.

Thus, in one embodiment, a method according to the present invention further includes administration of a therapeutic agent. Such a therapeutic agent is illustratively an anti-tumoral agent. Anti-tumoral agents are described, for example, in Goodman et al., Goodman and Gilman's The Pharmacological Basis of Therapeutics, 8th Ed., Macmillan Publishing Co., 1990.

Such anti-tumoral agents illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, droloxifene, dromostanolone, duazomycin, edatrexate, eflomithine, elsamitrucin, enloplatin, enpromate, epipropidine, epirubicin, erbulozole, esorubicin, estramustine, estramustine, etanidazole, etoposide, etoposide, etoprine, fadrozole, fazarabine, fenretinide, floxuridine, fludarabine, fluorouracil, fluorocitabine, fosquidone, fostriecin, fulvestrant, gemcitabine, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-I a, interferon gamma-I b, iproplatin, irinotecan, lanreotide, letrozole, leuprolide, liarozole, lometrexol, lomustine, losoxantrone, masoprocol, maytansine, mechlorethamine hydrochlride, megestrol, melengestrol acetate, melphalan, menogaril, mercaptopurine, methotrexate, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, puromycin, pyrazofurin, riboprine, rogletimide, safingol, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine sulfate, vindesine, vindesine, vinepidine, vinglycinate, vinleurosine, vinorelbine, vinrosidine, vinzolidine, vorozole, zeniplatin, zinostatin, zoledronate, and zorubicin. Each of these may be delivered in conjunction with a method and/or compositions according to the present inventions as a pharmaceutically acceptable salts, esters, amides, hydrates, and/or prodrug of any of these or other cytotoxins.

A second type of anti-tumoral treatment may also be administered in conjunction with PNP and APRT. For example, radiotherapy may be administered to a tumor before and/or after administration of PNP and APRT. Parameters for radiation therapy are known as exemplified in Washington, C. M. and Leaver, D. (Eds.), Principles and Practice of Radiation Therapy, C. V. Mosby; 2nd ed., 2003.

Further adjunctive therapeutic agents may be administered according to methods and in compositions of the present invention including analgesics, anesthetics, antibiotics, anti-inflammatory agents, nutritive supplements, vitamins, and other such agents beneficial to the subject.

### Compositions

A pharmaceutical composition for inhibiting a cell is provided which includes an expression vector including a nucleotide sequence encoding an adenine phosphoribosyltransferase. A pharmaceutically acceptable carrier is also included in such a pharmaceutical composition.

Optionally, certain embodiments of a pharmaceutical composition according to the present invention include an expression vector which includes a nucleotide sequence encoding a prokaryotic purine nucleoside phosphorylase.

A further embodiment is provided in which the expression vector including a nucleotide sequence encoding an adenine phosphoribosyltransferase and the expression vector including a nucleotide sequence encoding a prokaryotic purine nucleoside phosphorylase are the same vector, the nucleotide sequence encoding an adenine phosphoribosyltransferase and the nucleotide sequence encoding a prokaryotic purine nucleoside phosphorylase operably connected to a regulatory element in a bicistronic nucleic acid.

The term “pharmaceutically acceptable” as used herein is intended to mean a material that is not biologically or otherwise undesirable, which can be administered to an individual without causing significant undesirable biological effects or interacting in a deleterious manner with any of the other components of the pharmaceutical composition in which it is contained.

Depending on the intended mode of administration, the composition can be a pharmaceutical compositions in the form of solid, semi-solid or liquid dosage forms, such as, for example, tablets, suppositories, pills, capsules, powders, liquids, or suspensions, preferably in unit dosage form suitable for single administration of a precise dosage. Time release preparations are specifically contemplated as effective dosage formulations. The compositions will include an effective amount of the selected expression construct in combination with a pharmaceutically acceptable carrier and, in addition, may include other medicinal agents, pharmaceutical agents, carriers, or diluents.

For solid compositions, conventional nontoxic solid carriers include, for example, pharmaceutical grades of mannitol, lactose, starch, magnesium stearate, sodium saccharine, talc, cellulose, glucose, sucrose and magnesium carbonate. Liquid pharmaceutically administrable compositions can, for example, be prepared by dissolving or dispersing an active compound with optimal pharmaceutical adjuvants in an excipient, such as water, saline, aqueous dextrose, glycerol, or ethanol, to thereby form a solution or suspension. If desired, the pharmaceutical composition to be administered may also contain minor amounts of nontoxic auxiliary substances such as wetting or emulsifying agents, pH buffering agents, for example, sodium acetate or triethanolamine oleate.

For oral administration, fine powders or granules may contain diluting, dispersing, and/or surface active agents, and may be presented in water or in a syrup, in capsules or sachets in the dry state or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated.

Parenteral administration is generally by injection. Injectables can be prepared in conventional forms, either liquid solutions or suspensions, solid forms suitable for solution or prior to injection, or as suspension in liquid prior to injection or as emulsions.

Further examples and details of pharmacological formulations and ingredients are found in A. R. Gennaro, Remington: The Science and Practice of Pharmacy, Lippincott Williams & Wilkins, 20th ed. (2003); L. V. Allen, Jr. et al., Ansel's Pharmaceutical Dosage Forms and Drug Delivery Systems, 8th Ed. (Philadelphia, Pa.: Lippincott, Williams & Wilkins, 2004); J. G. Hardman et al., Goodman & Gilman's The Pharmacological Basis of Therapeutics, McGraw-Hill Professional, 10th ed. (2001). In addition, detailed information concerning materials, equipment and processes for preparing various dosage forms may be found in Pharmaceutical Dosage Forms: Tablets, eds. H. A. Lieberman et al. (New York: Marcel Dekker, Inc., 1989), and in L. V. Allen, Jr. et al., Ansel's Pharmaceutical Dosage Forms and Drug Delivery Systems, 8th Ed. (Philadelphia, Pa.: Lippincott, Williams & Wilkins, 2004).

### Administration of Compositions and Substrates

A pharmaceutical composition according to the present invention and/or substrate is administered by a route determined to be appropriate for a particular subject by one skilled in the art. For example, a composition and/or substrate is administered orally, parenterally (for example, intravenously), by intramuscular injection, by intraperitoneal injection, intratumorally, or transdermally. Intratumoral injections may be a single injection or, preferably, multiple passes in multiple locations within the tumor. Intratumoral instillation or infusion methods may also be used.

The exact amount of composition and/or substrate required will vary from subject to subject, depending on the age, weight and general condition of the subject, the severity of the disease that is being treated, the location and size of the tumor, the particular compound used, its mode of administration, and the like. An appropriate amount may be determined by one of ordinary skill in the art using only routine experimentation given the teachings herein. Generally, dosage will preferably be in the range of about 0.5-500 mg/m², when considering 5′-methyl(talo)MeP-R as a substrate for example, or a functional equivalent. For viral vectors, dosage is generally in the range of 5×10³-5×10¹⁰ pfu, depending on the size and location of the tumor, as well as the type of virus.

The formula of Freireich et al., Cancer Chemother. Rep., 50:219 244, (1966) can be used to determine the maximum tolerated dose of substrate for a human subject. For example, based on systemically administered dose response data in mice showing that a dose of 200 mg per kg per day of 5′-methyl(talo)MeP-R for 3 days (3 doses total) was well tolerated, a human dosage of 600 mg 5′-methyl(talo)MeP-R/m² was determined according to the formula: 200 mg/kg times 3=600 mg/m². This amount or slightly less should be tolerated in humans with minimal side effects. Furthermore, it is understood that modes of administration that permit the substrate to remain localized at or near the site of the tumor will be effective at lower doses than systemically administered substrates.

A subject having a tumor cell to be inhibited according to the present invention is a human or other mammal, illustratively including rodents, cats, dogs, rabbits, horses, cows, pigs, sheep and non-human primates.

Compositions and methods according to the present invention are described primarily herein with reference to prokaryotic PNPs and particularly with reference to E. coli PNPs. However, prokaryotic enzymes which are capable of cleaving purine containing nucleoside analog substrates to generate a substrate for APRT enzymatic activity and generation of a cytotoxic compound are considered within the scope of the present invention. Such prokaryotic enzymes include various prokaryotic hydrolases and phosphorylases as disclosed in U.S. Pat. No. 6,491,905. Further, various prokaryotic hydrolases and phosphorylases suitable for use in methods and compositions according to the present invention as well as methods and compositions for use of prokaryotic hydrolases and phosphorylases, particularly E. coli PNP, in inhibiting tumors are described in U.S. Pat. Nos. 5,552,311; 6,017,896; 6,491,905; 6,605,281; 6,958,318 and 7,037,718; and in U.S. Patent Application Publication Nos. 20050214901; 20040204375; 20030228576; 20030134819; and 20030077268.

Embodiments of inventive compositions and methods are illustrated in the following examples. These examples are provided for illustrative purposes and are not considered limitations on the scope of inventive compositions and methods.

### EXAMPLES

### Example 1

### Cloning of APRT

A cDNA encoding full length human APRT is isolated, the cDNA encoding the APRT protein of SEQ ID NO 1. In this example, cDNAs are synthesized from HeLa total RNA using an RNeasy kit from Qiagen and used in a PCR reaction to amplify APRT cDNA. FIG. 3 shows a 543 by band on a gel which is a PCR product encoding full length human APRT. The PCR products are cloned into a pCR4-Blunt Topo vector, available commercially from Invitrogen. The resulting clone is verified by DNA sequencing.

This 543 by APRT coding sequence may be amplified with primers to introduce restriction sites, such as NcoI and XhoI, into the product suitable for cloning into an expression vector. FIG. 4 shows an example of an expression vector according to the present invention containing a DNA sequence encoding human APRT.

### Example 2

### Cloning of E. coli PNP

A bacterial PNP-encoding sequence is inserted into an expression vector. In one example, E. coli (strain, JM101) chromosomal DNA template is obtained using the method described in N. J. Gay, J. Bacteriol., 158:820-825 (1984). Two PCR primers GATCGCGGCCGCATGGCTACCCCACACATTAATGCAG and GTACGCGGCCGCTTACTCTTTATCGCCCAGCAGAACGGA-TTCCAG are used to define the full length coding sequence of the E. coli DeoD gene and to incorporate NotI sites at both 5′ and 3′ ends of the desired product. After 30 cycles of amplification (94° C.×1 minute denaturation, 50° C.×2 minute annealing, and 72° C.×3 minute elongation using 1 ng template, 100 microliters of each primer in a 100 microliter reaction mixture containing 2.5 units taq polymerase, 200 micromolar each dNTP, 50 mM KCl, 10 mM Tris Cl (pH 8.3), 1.5 mM MgCl2 and 0.01% gelatin (weight/vol)), a single PCR product of the predicted size is obtained. This product is extracted with phenol/chloroform, precipitated with ethanol, digested with NotI, and gel purified.

The amplified bacterial PNP sequence is inserted into a eukaryotic expression vector. In order to obtain a vector capable of directing eukaryotic expression of E. coli PNP, the LacZ gene is excised from pSVB (Clontech, Palo Alto, Calif.) by digestion with NotI, the vector backbone was dephosphorylated (calf intestinal alkaline phosphatase, GIBCO BRL, Gaithersburg, Md.) and gel purified as above. The PNP insert, prepared as above, is then ligated into the NotI ends of the plasmid backbone in order to create a new construct with PNP expression controlled by the SV-40 early promoter. Identity of recombinants and orientation of inserts are confirmed by restriction mapping and by reamplification of the full length insert from recombinant plasmid using the primers described above. This procedure yields an expression construct SV-PNP.

### Example 3

### Generation of Herpesvirus Expression Vectors

HSV vectors exhibit tumor specificity in vivo by selectively targeting the proliferating cells in a growing tumor mass. Certain HSV vectors have been used previously in the clinic to examine glioma therapy (for micrometastatic disease) and by regional administration to liver for treatment of metastatic colon cancer Shah, A. C., et al., 2003, J. Neuro-Oncol. 65: 203-226; Markert, J. M., et al., 2000, Gene Therapy 7: 867-874; and Rampling, R., et al., 2000, Gene Therapy 7: 859-866.

Sequences encoding PNP and/or APRT are inserted into an HSV targeting plasmid. The targeting plasmids are separately co-transfected into rabbit skin cells (RSC) with C101 viral DNA that is isolated and digested with the restriction enzyme PacI (which removes a GFP expression cassette from the virus). After co-transfection, the viral genome is reconstituted by homologous recombination and viral plaques selected, propagated, and subjected to two additional plaque purifications on Vero cells. Candidate virus clones are confirmed by Southern blot hybridization of restriction enzyme-digested viral DNAs. Detection is performed with alkaline phosphatase, using Gene Images AlkPhos Direct DNA labeling system, Amersham-Pharmacia Biotech, Piscataway, N.J.

An egr-1 promoter is an example of a promoter included in PNP and APRT HSV constructs for expression of PNP and/or APRT.

### Example 4

### Generation of Enhanced Expression HSV Vectors

APRT and/or PNP-expressing γ134.5-HSVs are constructed using a modification of a technique described by Krisky, D. M., et al., 1997, Gene Therapy 4: 1120-1125.

A γ134.5-, tk+ HSV, termed C101, is used. C101 is an HSV vector derived from R3616, described in detail in Whitley, R. J., et al., 1993, J Clin Invest 91(6):2837-43, which contains a CMV-driven transgene cassette introduced within the UL3/UL4 intergenic region. This virus represents a substantial improvement over earlier generation HSV, since 1) it encodes a more active transgene regulatory element, and 2) the UL39 region does not contain a transgene insertion. An intact UL39 region allows improved intratumoral spreading and distribution.

PNP and APRT HSVs are constructed in which the PNP and/or APRT coding sequence is subcloned from a shuttle plasmid into an HSV targeting plasmid. with sequences homologous to those flanking the UL3/UL4 intergenic region. For PNP, the pCA13-wtPNP is used to excise a PNP encoding sequence which is inserted into an HSV targeting plasmid (pCK1037). The resulting targeting plasmid containing PNP, pHN001, is co-transfected into rabbit skin cells (RSC) with C101 viral DNA that had previously been isolated and digested with the restriction enzyme PacI to removes a GFP expression cassette from the virus. Similarly, an APRT encoding sequence is inserted into an HSV targeting plasmid and cotransfected as described.

After co-transfection, the viral genome is reconstituted by homologous recombination and viral plaques are selected, propagated, and subjected to two additional plaque purifications on Vero cells. Candidate virus clones are confirmed by Southern blot hybridization of restriction enzyme-digested viral DNAs (detection performed with alkaline phosphatase; Gene Images AlkPhos Direct DNA labeling system, Amersham-Pharmacia Biotech, Piscataway, N.J.). This allows exclusion of any gross rearrangements that otherwise might have occurred during viral construction. Viral DNA is isolated from these positive candidates, and the PNP or APRT gene inserts sequenced to rule out subtle changes that would prevent production of authentic enzyme.

Assay for functional enzyme may also be performed to verify correct constructs.

### Example 5

### Generation of Adenovirus Expression Vectors

Adenovirus (Ad) construction typically requires a two plasmid transfection that includes 1) an Ad shuttle plasmid and 2) a genomic plasmid, as described below.

E1 Shuttle Plasmid

An E1 shuttle vector is provided to allow replacement of the adenoviral E1 promoter with an E2F1 responsive element. The vector is established from a commercially available Ad vector (pShuttle, Strategene) that lacks a large portion of E1. Nucleotides encoding the first segment of E1A and upstream regions (positions 191-1339 of the Ad5 genome) are excised from pXC1 (Microbix, Canada) with BsrGI and XbaI and ligated into corresponding sites in pShuttle. The MA promoter in pShuttle is next removed by restriction digestion, and replaced with a PCR product containing an E2F1 regulatory element and an 8 amino acid deletion of the E1A CR2 domain (deltaCR2), as described in Johnson, L., et al., 2002, Cancer Cell, 1(4):325-37. The deltaCR2 modification further limits the ability of the virus to overcome the G1-S checkpoint in normal cells, and renders the vector less able to replicate and spread, except in tumors. The shuttle vector is completed by adding 4426 bp of adenoviral genomic DNA excised from pXCI with XbaI and Bst1107I. The identity of deltaCR2, the E2F1 promoter and all other key regions of the shuttle are confirmed by DNA sequencing.

Ad Genomic Plasmid

In order to establish an Ad genomic plasmid for co-transfection, an E3/E4 plasmid is first constructed to allow 1) regulation of adenoviral E4 expression by the E2F1 promoter, and 2) insertion of PNP expression cassettes into the E3 region. The pAdEasy-1 adenoviral plasmid (Stratagene) is digested with EcoRI and the large fragment self-ligated to obtain a simplified plasmid that contains E3 and E4 of the Ad5 genome. In order to place E4 expression under E2F1, PCR is performed to generate an Ad fragment (nt 35463-end) with new Spe1 and Xho1 restriction sites surrounding the E4 promoter, (positions 35,376 and 35,816 respectively). This is ligated into the corresponding region of the E3/E4 plasmid. An E2F1 promoter flanked by SpeI and XhoI is generated by PCR, and used to replace the E4 promoter, just upstream of E4. All key regions and PCR inserts are verified by DNA sequencing.

Various regulatory elements may be included to drive expression of the PNP and/or APRT transgenes in such constructs, illustratively including CMV and E2F1.

For example, a nucleic acid sequence encoding E. coli PNP or APRT is cloned into the E3 region of pAB27 (Microbix). A Clal/BamHI digest is used to excise the CMV promoter and allow substitution of a PCR amplified E2F1 regulatory element. The E2F1 promoter is confirmed by DNA sequencing. PNP and APRT gene expression cassettes, such as CMV driven and E2F1 driven PNP and APRT gene expression cassettes, are cut out of pAB27 by digestion with EcoRI and HpaI and cloned into the corresponding sites of the E3/E4 plasmid described above. A DNA fragment containing these sequences (as well as E2F1 driven E4) is then excised from the E3/E4 plasmid with SrfI and Pad and cloned into the corresponding sites of pAdEasy-1 (Stratagene) to establish the final genomic plasmid.

Production of Recombinant Adenovirus

The pAdEasy-1 bacterial recombination system (Stratagene) is used to isolate recombinant adenovirus. The E1 shuttle plasmid is linearized with PmeI, and cotransformed into BJ5183 cells along with the modified pAdEasy described above.

Transformants are selected by kanamycin resistance, and recombinants verified by restriction digest. Once a recombinant is identified, it is produced in bulk using recombination-deficient bacteria (XL10-Gold®). Purified recombinant Ad plasmid DNA is digested with Pac I to expose the inverted terminal repeats (ITR) and then used to transfect HEK293 cells as described in Bristol, J. A., et al., 2003, Mol Ther. 7(6):755-64.

When viral plaques appear on a lawn of cells, the tissue culture supernatant is used as source of recombinant virus. Identity is verified by PCR, together with DNA sequence analysis of all key elements or modifications.

### Example 6

### Conditionally Replicating Adenoviral Vectors for E. coli PNP and Human APRT Expression

Conditionally replicating adenoviral vectors allow spread of viruses expressing the transgenic PNP and APRT through tumor masses in vivo. Adenoviral vectors that do not injure normal, mitotically quiescent, tissues but confer selective lysis in cancer cells have a deletion of the Ad E1B 55K region as described in detail in McCormick, F., 2003 Cancer Biol Ther., 2(4 Suppl 1):S157-60; Linke, S. P., 1998, Nature 395: 13-15; Rothmann, T., et al., 1998, J. Virol. 72: 9470-9478; Hann, B., and Balmain, A., 2003, J. Virol. 77(21): 11588-95; Wadler S., et al., 2003, Clin Cancer Res. 9(1):33-43; Jakubczak, J. L., et al., 2003, Cancer Res. 63(7): 1490-9; and Post, L. E., 2002, Curr Opin Investig Drugs 3(12):1768-72.

In a further option, a virus constructed to encode a deletion within the E1A-CR2 region is used which precludes replication in normal cells by maintaining pRB activity despite the presence of the virus while robustly replicating in human tumor cell types including carcinomas of the lung, colon, pancreas, prostate, breast, cervix, osteocarcinomas, and head and neck cancers, in a fashion dependent on E2F1 over-expression. Oncolytic adenoviruses encoding E. coli PNP and/or APRT are provided that replicate specifically in tumor cells. E. coli PNP and/or APRT are cloned into the E3 region of a virus otherwise identical to the one described in Johnson, L., et al., 2002, Cancer Cell, 1(4):325-37.

### Example 7

### Lentivirus Expression Constructs

Lentivirus constructs are generated as described in Bennett, E. M., et al., 2003, Chemistry and Biology 10, 1173-1181; Hong, J. S., et al., 2004, Cancer Research 64, 6610-6615; and Bharara, S., et al., 2005, Human Gene Therapy 16:339-347. Plasmids for use in generating lentivirus constructs, such as the envelope-coding plasmid pMD.G; the packaging plasmid pCMVDR8.91, which expresses Gag, Pol, Tat, and Rev; the transfer vector plasmid, without the PNP or APRT encoding sequences may be obtained from Trono et al., Lausanne, Switzerland. Analogous plasmids are obtained commercially or synthesized.

Briefly, an E. coli PNP encoding nucleic acid sequence is PCR amplified by primers 5′-ggatccaccatggctaccccacacattaatg-3′ (Baran site and ATG underlined) and 5′cctcgagtcactctttatcgcccagcag-3′ (XhoI site underlined). The resulting product is subcloned into ZeroBlunt vector (Invitrogen, Carlsbad, Calif.). After digestion with BamHI and XhoI, the luciferase gene in the pHR′CMVLuc W Sin-18 lentivirus vector is replaced with E. coli PNP. Correct insertion is verified by sequencing and by transfection into cells followed by assay for active E. coli PNP enzymatic activity. A CMV promoter is included in this construct.

A DNA mixture containing 5 micrograms of envelope-coding plasmid pMD.G; 15 micrograms of the packaging plasmid pCMVDR8.91, which expresses Gag, Pol, Tat, and Rev; and 20 micrograms of transfer vector plasmid is used for calcium phosphate transfection of one 10 cm dish of 293T cells. Replication-deficient viral particles encoding E. coli PNP are collected from tissue culture supernatant after transfection and may be concentrated for further use.

Similarly, a human APRT lentivirus expression vector is generated essentially as described above using an APRT encoding nucleic acid sequence such as the 543 by insert described above, modified as desired to introduce appropriate restriction sites.

Verification of correct insertion is assayed by sequencing and by transfection into cells followed by assay for active APRTenzymatic activity.

### Example 8

### Bicistronic Constructs

E. coli PNP and human APRT may be encoded on separate expression constructs which are transferred into a cell for co-expression of the proteins. Alternatively, a bicistronic vector is constructed for such co-expression. For example, E. coli PNP and human APRT gene may be inserted into a bacterial plasmid construct and/or a viral vector.

A polycistronic adenovirus transfer vector, pShuttle-IRES-hrGFP-1, commercially available from Stratagene, provides strong expression in adenovirus from two independent transgenes. The APRT gene is isolated from the above-described pCR4Blunt-APRT construct (FIG. 4) by digestion with NcoI and EcoRI, and then cloned into the NcoI and EcoRI sites of an IRES containing transfer vector designated pTM-APRT). This plasmid is digested with KpnI and SpeI and cloned into KpnI and XbaI sites of the pShuttle-IRES-hrGFP1 vector. XbaI and SpeI share compatible overhangs. The resulting plasmid contains the APRT gene in place of hrGFP. The PNP gene is cloned into the Nod site of the plasmid by isolating PNP from NotI digested pSV-PNP (Sorscher, E. J., et al., 1994, Gene Therapy 1:233-238). Subsequent adenoviral construction using the bicistronic cassette encoding APRT and PNP is as described above, and modified for oncolytic adenovirus.

A similar strategy is adapted for cloning into HSV. The EMCV IRES sequence in HSV allows for excellent bicistronic expression. The HSV can accommodate at least 9 kb with little impact on viral replication. A cassette containing both APRT and PNP trangenes may be inserted into the UL3/UL4 region so as to place one copy of each gene into the virus. Further details of cloning into HSV are described in Parker, J. N., et al., 2000, PNAS, 97(5): 2208-2213.

### Example 9

### Assay for PNP Activity

E. coli PNP activity may be measured by HPLC. For example, cells infected with an E. coli expression construct are used to prepare crude cell extracts as described in Sorscher, E. J., et al., 1994, Gene Ther., 1:233-238; and Parker, W. B., et al., 2003, Cancer Gene Ther., 10:23-29. The extracts are incubated with various concentrations of MeP-dR, and the formation of product is measured by HPLC analysis of the reaction mixture as described in Gadi, V. K., et al., 2000, Gene Ther., 7:1738-1743. Activity is expressed as PNP units where one unit represents 1 nmol MeP-dR-converted/mg cell extract/hour.

### Example 10

### Assay for APRT Activity

Cell-free extracts, at a concentration that yields linear increases in AMP formation, are incubated with 50 mM Tris (pH 7.4), 5 mM MgCl₂, 100 micromolar [³H]adenine, and 1 mM phosphoribosyl pyrophosphate at increasing time intervals, e.g. 30, 60, 90, 120 minutes, at 37° C. The reaction is stopped by applying the reaction mixture to DE-81 disks, a type of anion exchange disk, and washed with buffer to remove the substrate, adenine. The product of the reaction, [³H]AMP, adheres to the filter under these conditions. The disks are washed with ethanol, dried, and counted for radioactivity. Alternatively, the reaction mixture can be analyzed by strong anion exchange HPLC to separate product from the substrate as described below.

### Example 11

### Extraction and Analysis of the Acid-Soluble Nucleotide Pool

SAX HPLC as described in Someya H, et al., 2006, Cancer Chemother. Pharmacol., 57(6):772-80; Parker W B, et al., 2004, 84: 327-336; Parker W B, et al., 2000, 60: 1925-1932; Parker W B, et al., 1999, Mol. Pharmacol., 55: 515-520; Parker W B, et al., 1998, Biochem. Pharmacol., 55: 1673-1681; Parker, W B, et al., 1993, Antimicrob. Agents Chemother., 37: 1004-1009; and Parker W B, et al., 1991, Cancer Res., 51: 2386-2394, is used to evaluate the intracellular metabolism of prodrug. Briefly, cells incubated with radiolabeled analogs are collected by centrifugation and resuspended in ice-cold 0.5 M perchloric acid. The samples are centrifuged at 12,000×g for 20 minutes, and the supernatant fluid removed and neutralized with 1 M potassium phosphate (pH 7.4) and 4 M KOH. KClO₄ is removed by centrifugation, and a portion of the supernatant fluid is injected onto a Partisil-10 SAX column (Keystone Scientific Inc, State College, Pa.). Elution of the nucleotides is accomplished with a 50-min linear gradient from 5 mM NH₄H₂PO₄ (pH 2.8) to 750 mM NH₄H₂PO₄ (pH 3.7) buffer with a flow rate of 2 ml/min. The natural nucleotides are detected by measurement of the UV absorbance at 260 nm, and the radioactive acid-soluble metabolites are detected by counting 1-minute fractions eluting from the column.

### Example 12

### Reverse Phase HPLC to Measure Prodrug and Product

Acid soluble extracts are prepared as described above. Samples are injected onto a 5 micron BDS Hypersil C-18 column (150×4.6 mm) (Keystone Scientific Inc., State College, Pa.), and the substrates and products separated using 5% acetonitrile in 50 mM ammonium dihydrogen phosphate buffer (pH 4.5) as mobile phase at a flow rate of 1 ml/minute. The inventors have successfully used this system (changing only the percentage of acetonitrile from 1 to 50%) to separate 105 pairs of substrates and products as described in Secrist, J. A., et al., 1999, Nucleosides and Nucleotides 18: 745-757.

Fractions are collected as they elute from the column, and the radioactivity in each is determined.

### Example 13

### Antibody-Based Detection

In addition to functional enzyme assays and sequencing methods to verify functional expression vectors, detection, localization, characterization and quantitation of PNP and APRT products of expression vector expression may be assessed using antibodies specific for PNP or APRT.

No antibodies against E. coli PNP have been available previously for localization or other studies. We recently used purified, functional E. coli PNP and techniques such as described in Sorscher, E. J., et al., 1992, American Journal of Physiology 262, C136-C147; and Demolombe, S., et al., 1996, Gene Therapy 3, 685-694, to develop a panel of monoclonal and polyclonal antibodies specifically binding E. coli PNP.

Antibodies are raised against purified recombinant E. coli PNP. E. coli PNP expressed by Ad-PNP infected HeLa cells is detectable both with a generated mouse monoclonal antibody and rabbit polyclonal antibody.

The generated mouse monoclonal and rabbit polyclonal antibodies may be used to detect expressed E. coli PNP by immunoprecipitation, immunoblot and ELISA.

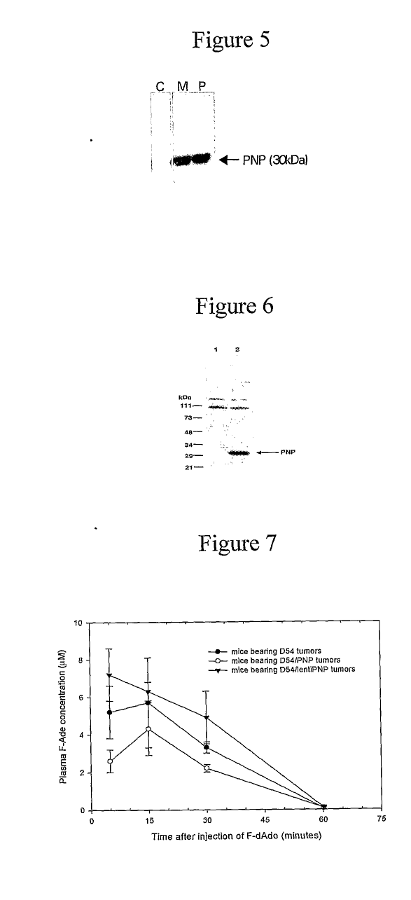

Immunoprecipitation from Ad-PNP infected HeLa cell lysates is detected using these antibodies as shown in FIG. 5. The PNP protein is labeled with 35S-Met following infection, and immunoprecipitated using protein G agarose beads. C: uninfected control, M: mouse monoclonal antibody, P: rabbit polyclonal antibody.

Western blot of glioma tumors transduced with E. coli PNP is shown in FIG. 6. Flank tumors are injected with a replicating retrovirus (RCR) encoding E. coli PNP. Western blotting of PNP-transduced tumor lysates (at the 6 week time point) is shown (lane 2), with control (non-PNP transduced) tumor lysates taken on the same day for comparison (lane 1).

### Example 14

### Tumor Models—In Vitro and In Vivo

Various tumor models are used to assess the effects of compositions and methods according to the present invention. In vitro models include culture models of tumor cell growth and survival. Exemplary in vitro models include culture of D54 human glioma tumor cells.

Cells used for in vitro models may be transfected and infected with various expression constructs in conjunction with administration of substrate for PNP and/or APRT. Survival assays include assays such as MTT assay or crystal violet staining. Cell extracts may be used to assess PNP and/or APRT expression as well as total nucleic acid and/or protein expression.

Cells in vitro may also be used to demonstrate effects on bystander cells.

In vivo tumor models include any of various models standard in evaluation of chemotherapeutics. For example, introduction of tumor cells into mice allows assessment of anti-tumoral compositions and methods.

For example, in one in vivo tumor model, tumors are established in animals by subcutaneous implantation of about 10⁷ cells into the axillary region of 6-10 mice. Athymic (or scid) mice are used for models of certain human tumors including cells such as D54, HCT-15, and SR475 head/neck. Syngeneic models are also used. For instance, mouse tumors are established using Colon 26 and G26 glioma cells.

The resulting solid tumors are measured twice weekly in two dimensions and the mass calculated using the formula for a prolate ellipsoid, [length (mm)×width² (mm)]/2=mg, assuming unit density. The data will permit the determination of a tumor volume doubling time, % “no takes”, and % spontaneous regressions.

Further details of tumor xenografts and their analysis are described in Alley, M. C., et al., 2004, Human tumor xenograft models in NCI drug development, In: B. A. Teicher (ed.), Anticancer Drug Development Guide, 2nd Edition, pp. 125-152, Totowa, N.J.: Humana Press.

### Example 15

### Glioma Models

For intracranial glioma models, 5×10⁵-1×10⁶ tumor cells are injected into the right frontal lobe using the following coordinates: 1 mm anterior and 1.5 mm lateral relative to the bregma, and 2.8 mm deep into the brain. At a later time point mice are stereotactically injected with PBS vehicle or virus encoding PNP and APRT in 10 microliters. Intracranial inoculations are conducted under anesthesia (ketamine, xylazine) and with post-procedural tylenol in water. Intracranial tumors may be evaluated in various ways, including excision and measurement, as well in situ evaluation using imaging for instance. An in situ imaging method is described herein.

### Example 16

### Virus Administration to Flank Tumors

Cancers established in the flanks of test animals are injected with replicating HSV or Ad vector including sequences encoding PNP, APRT or PNP and APRT. In general, a tumor having an approximate weight of 300 mg is injected with 10⁷-10¹⁰ pfu daily for 1-3 days. Virus is typically inoculated intratumorally in a volume of 50-100 microliters along four needle tracks (200-300 milligram tumors).

Measurements including E. coli PNP activity, antibody localization of PNP protein, APRT activity and antibody localization of APRT protein are determined at suitable intervals, such as days 1, 3, 5, 9, and 15 after virus administration and longer time points if necessary or desirable.

Various viral vectors may be used in this model, including conditionally replicating viruses (HSV, Ad) and conventional, non-replicating constructs (adenovirus) for example.

Prodrug is administered to animals at various times following virus injection.

Controls include treatment with no virus or prodrug, virus without prodrug, or prodrug without virus. Further controls include administration of viruses expressing a control protein, such as GFP or luciferase.

It is preferred that prodrug is administered at a time when PNP activity and vector spreading are optimized, so that effects of prodrug will provide the greatest synergy. Brief courses of E. coli PNP prodrugs mediate anti-tumor effects with replicating PNP viruses such as HSV, replicating vaccinia, and replicating retrovirus as described in Bharara, S., et al., 2005, Human Gene Therapy 16:339-347; and Puhlmann, M., et al., 1999, Human Gene Therapy 10, 649-657. The synergy between permissively replicating vectors and suicide genes is expected to be greatest if prodrug dosing occurs when intratumoral vector spread is maximal so as to ablate both tumor cells and further intra- or extratumoral dissemination of the vector. Prodrug is administered at a time suitable for robust ablation of tumor tissues, based on the expression endpoints described above.

### Example 17

### Models of Colonic Carcinoma Metastatic to Liver

Metastatic colon cancer is a devastating disease and syngeneic colon cancer model provide a valuable assessment of inventive compositions and methods. The murine colon 26 model in which portal vein tumor cell inoculations are used to seed livers of congenic Balb C mice is illustrated in this example. In the colon 26 model, liver weights, number of metastases, survival, etc. are evaluated as endpoints as described in detail in Nishikawa, M., et al., 2004, Clin Exp Metastasis. 21, 213-21; and Hayashi, S., et al., 1999, Cancer Gene Ther. 6, 380-4. To determine the effect of compositions and methods according to the present invention on the number of metastatic foci, mice receive an intraportal vein injection of about 1×10⁵ colon 26 tumor cells. For each study, twenty mice may be included in a control group and six mice in each of the treatment arms. Adenovirus-PNP and/or adenovirus-APRT (replication deficient; initially dosed at 5×10⁷ to 5×10⁹ pfu), replicating Ad-PNP and/or Ad-APRT (5×10⁶ to 5×10⁸ pfu) (Vranken Peeters M J, et al., 1996, Biotechniques. 20(2):278-85) or HSV-PNP and/or HSV-APRT (e.g. 3×10⁵ to 3×10⁶ pfu) are injected into the portal vein followed by a 2-4 day interval to allow for E. coli PNP and human APRT expression. In general, the lower doses of HSV are considered optimal since larger doses may saturate receptors and pass directly through the hepatic compartment.

A prodrug is administered following inoculation with virus. Dosage and administration schedule depend on a number of factors assessed in the specific context, such as animal weight, tumor weight, and tumor type, for example. In one example, F-araAMP is administered intraperitoneally (ip) at 25-100 mg/kg/dose which is given 5 times a day for 3 consecutive days. Other dosing and administration options include 50 mg/kg ip q2h×5, qld×3; 125-175 mg/kg q1D ×3-q4H ×3; or 200-300 mg/kg q1D ×3. Controls include 1) no virus or prodrug administration, 2) prodrug only, 3) PNP virus only and 4) APRT virus only.

Starting on day 10 after viral inoculation, a group of 3-4 control animals is sacrificed every two days to assess the development of metastatic foci in the liver. When an optimum number of foci (n=100 to 200) are present (usually around 15 days) the remaining control animals and the treated animals are sacrificed, and the livers of all animals are excised, weighed, and assayed for transgene expression, viral recovery, or placed in fixative for at least 24 hours prior to counting the metastatic foci. The tumors and surrounding liver parenchyma are analyzed for PNP and/or APRT immunofluorescence. Experiments are also conducted to determine the effect of treatment on life span. These latter studies are performed as described above except that only ten mice are in the control group and no animals are sacrificed, but survival checked daily.

Since vectoring systems described in this project (HSV and oncolytic adenovirus) are reported to specifically target metastatic colon cancer cells as described in Shah, A. C., et al., 2003, J. Neuro-Oncol. 65, 203-226; and Johnson, L., et al., 2002, Cancer Cell 1(4), 325-37, both may be used in the model described above.

### Example 18

### In Vivo Evaluation of Intracranial Glioma Tumors

Therapeutic efficacy of PNP and APRT expression in intracranial glioma tumors may be illustrated using HSV and adenovirus constructs in human (D54) and murine (G26) glioma models. G26 gliomas are studied in congenic (B6D2F1) mice. The glioma cell lines are transduced with a firefly luciferase expression construct to monitor tumor growth and response to therapy by bioluminescence imaging.

In such a model of in vivo demonstration of PNP and APRT anti-tumoral activity, scid mice are implanted intracranially with 5×10⁵ to 1×10⁶ luciferase-expressing human glioma cells. Animals may be implanted with an electronic biotag to permit unequivocal identification. Over the next 10-55 days, mice may be imaged to detect firefly luciferase expression and tumor growth is thereby monitored. Images are collected on mice oriented in the same position at a specified time, such as 10 minutes, following intraperitoneal injection of a specified amount of luciferin. In certain experiments, 2.5 milligrams of beetle luciferin are injected. During imaging, mice are maintained under 1.5% enflurane/oxygen gas anesthesia at 37° C. Image acquisition times are in the range of 20 seconds to 10 minutes. Data acquisition software ensures that no pixels are saturated during image collection. Light emission from the tumor regions (relative photons/sec) may be quantified using software provided by Xenogen.

After tumor induction and growth to a desired size, viruses expressing PNP and APRT, either separately or bicistronically, are injected intratumorally. For example, intracerebral administration includes initial doses of about 1-5×10⁶ pfu HSV and/or 1×10⁶-5×10⁷ pfu adenovirus in 5-10 microliters.

A prodrug is administered after inoculation with virus. For example, F-araAMP may be administered in various amounts on various schedules such as about 25-100 mg/kg q 2h ×5, q1d ×3d; 125-175 mg/kg q1D ×3-q4H ×3; or 200-300 mg/kg q1D ×3. The prodrug may be administered by injection directly into the intracranial tumor. Optionally, a pump may be implanted to deliver an intratumoral dose of a prodrug. A prodrug may also be administered systemically.

Controls include untreated animals, vector without prodrug treated animals, and prodrug without vector treated animals. Control and experimental samples may be harvested at various times such as 1, 3, 5, 9 and 13 days. The samples are assayed for endpoints including antibody localization and/or in situ hybridization for the fraction and distribution of PNP-expressing cells.

A prodrug, such as F-araAMP, is administered 2-3 days after virus or later time points as guided by PNP expression assays. All mice are imaged twice weekly to follow changes in tumor growth by bioluminescence.

### Example 19

### Statistical Analyses and Considerations in Therapeutic Studies

The inventor's experience with the intracranial (i.c.) inoculation of tumor cells has shown that host death in each cohort of properly transplanted, mock-treated mice occurs within a predictable and narrow window, providing highly uniform data for determining statistically significant treatment differences. Survival Analysis (log-rank tests) are performed following i.c. inoculation to determine any important differences observed between treatment groups. Analyses of process variables is not possible when death is used as the outcome variable of interest. In the flank and colonic models described here, data is typically uniform and normal and can be analyzed by parametric methods (e.g. Student's t-Test). Any data which does not fit a normal distribution are subjected to standard, non-parametric type analyses (e.g. Mann-Whitney rank sum test). If tumors do not reach a designated evaluation point, life table analyses are used (SYSTAT Version 7.0).

Descriptive statistics (mean, median, and standard deviation) are applied to in vitro continuous data, HPLC based PNP or APRT activity measurements, including modified PNP's, and relative APRT mRNA levels. Tests of statistical significance include paired t-tests (Sigma-stat software). All in vitro studies are performed in a carefully paired fashion (same day, same cell passage). A p value (α) of less than or equal to 0.05 is considered statistically significant.

### Safety Analysis and Endpoints

Tumor regressions in flank and CNS models with PNP/F-araAMP have not indicated significant toxicity (as judged by weight loss or lethality) and have improved animal longevity.

### Example 20

### Overexpression and Detection of APRT

D54 human glioma cells are used to examine effects of PNP expression and APRT overexpression. In this example, lentivirus constructs are used to infect the cells. The transduction efficiency achieved at MOI 1-10 is typically sufficient to allow D54 PNP cells co-expressing APRT to be clonally expanded without a selectable marker. Recombinant expression of APRT in specific clones is assayed enzymatically and by RT-PCR. Quantitative or semi-quantitative RT-PCR is performed using a primer set specific for the particular vector that in order to distinguish vector derived mRNA from endogenous APRT message. Such assays may be performed on standard equipment such as an ABI Prism 7500 Sequence Detection System, Assays on Demand, Applied Biosystems. Semi-quantitative RT-PCR analysis and/or enzymatic assays described allow monitoring of APRT overexpression.

Primer sequences for assay of endogenous and lentivirus overexpressed APRT

include the following: endogenous APRT specific primers; 5′TGGCTCTTCGCACGCGGCCATGG 3′ (forward, initiation ATG codon in bold letters) and 5′ CACGCAGCCCAGTCCAAGCTCCT 3′ (reverse); lentivirus expressed APRT specific primers; 5′-TCTAGCTAGAGGATCCACCATGG 3′ (forward, lentiviral vector sequence underlined) and 5′-CGACCACCCTCTGTCCTGGCTCCA 3′ (reverse). The sizes of expected bands from RT-PCR are 271 by and 392 bp, respectively. D54-PNP tumors with and without APRT co-expression are compared for APRT mRNA and functional expression.

### Example 21

### In Vitro Evaluation of APRT Activity

Enzymatic activity of APRT and metabolism of F-araA are evaluated in paired cell lines that express E. coli PNP and which overexpress APRT. Radiolabeled F-araA is used, such as is commercially available from Moravek Biochemicals. Amounts of intracellular phosphorylated metabolites in cells is determined using techniques adapted from those described in Parker, W. B., et al., 1998, Biochem. Pharmacol., 55:1673-1681; Parker, W. B., et al., 2002, Cancer Gene Therapy 9, 1-7; Hughes, B. W., et al., 1995, Cancer Research, 55:3339-3345; and Gadi, V. K., et al., 2003, J. Pharm. Exper. Therap., 304: 1280-1284; and see FIG. 7, described in example 21 below).

Briefly, media samples are obtained and the amount of base, F-Ade, in the medium is assessed. Using these procedures increased accumulation of F-Ade within tumor cells, and a reduced amount of purine that is released extracellularly, a potential contributor to systemic toxicity, may be monitored.

### Example 22

### Experiments using Herpes Simplex Viruses for PNP and APRT Expression

Using HSV-PNP, a composition including 17 million pfu of the virus in 50 microliters is administered on day 16 following establishment of human glioma tumors in mice. HSV-PNP by itself slows growth of tumors following intratumoral inoculation, approximately 20 days delay to one tumor doubling. Intratumoral administration of MeP-dR (168 mg/kg daily×3 days) with HSV-PNP, improves tumor regressions and arrest of tumor growth in mice.

Tumors are infected with both HSV-PNP and HSV-APRT to further improve anti-tumoral response.

### Example 23

### In Vivo Generation and Clearance of F-Ade and Metabolism of F-AraAMP

Three different in vivo parameters are assessed in mice bearing tumors that express E. coli PNP and APRT; 1) the plasma half-life of prodrug and plasma levels of F-Ade that are generated in mice, 2) the amount of F-Ade metabolites that are associated with the tumors and other tissues, and 3) evidence of PNP, APRT, and viral delivery to tumors and extratumoral tissues.

For example, overexpression of APRT is intended to increase prodrug activation and augment accumulation of F-Ade in tumor tissues, while minimizing release of this agent into the circulation. Measurements of F-Ade and metabolites in tumors, blood, and tissues provide a biochemical test of this effect. Further, measurement of PNP and APRT levels and activity in a targeted tumor and various other tissues is performed to ascertain effectiveness of delivery and expression.

Measurements of generation and clearance of circulating F-Ade are made by HPLC for example. Radiolabeled prodrug is obtained from Moravek Biochemicals and injected into mice by the same route and dosage used in efficacy experiments for comparison. Plasma samples are removed at increasing time intervals after injection of prodrug and analyzed by reverse-phase HPLC for parent compound and base. Identification of prodrug and F-Ade is by HPLC as described above.

### Example 24

Monitoring release of F-Ade into blood following prodrug therapy. The experiment shown in FIG. 7 describes generation of F-Ade from the active PNP substrate, F-dAdo (Table 1).

For experiments shown in FIG. 7, sixteen mice are implanted subcutaneously with D54 parental, no PNP expression cells, D54/PNP MuLv, approximately 250 PNP units), or D54/lentiPNP, ˜426,000 PNP units tumor cells as described for efficacy evaluations. When tumors are approximately 200 mg the mice are injected intraperitoneally with 20 mg/kg 3H-F-dAdo. This dose of F-dAdo is the maximally tolerated dose of F-dAdo when given 5 times daily for 3 consecutive days. Four mice from each group are sacrificed 5, 15, 30, and 60 minutes after injection of F-dAdo, and plasma samples were obtained. The amount of F-Ade is determined in each plasma sample using reverse phase HPLC, as described.

There is no difference in the F-Ade plasma levels in mice bearing tumors with different levels of E. coli PNP activity, which is consistent with the fact that the MTD of F-dAdo is not reduced in mice bearing tumors that express E. coli PNP. Evaluation of F-dAdo with many vectors that express E. coli PNP shows no enhancement of toxicity due to intratumoral PNP expression as described in Parker, W. B., et al., 2002, Cancer Gene Therapy 9, 1-7; and Parker, W. B., et al., 1997, Human Gene Therapy 8, 1637-1644.

A similar experiment is conducted with F-araAMP. However, no F-Ade is detected in the plasma of mice bearing tumors expressing E. coli PNP that are injected with 100 mg/kg F-araAMP, which is the maximally tolerated dose of F-araAMP when given 5 times daily for 3 consecutive days. The limit of detection in these experiments with fludarabine is approximately 1 micromolar. As with F-dAdo, the MTD of F-araAMP is not reduced in mice bearing tumors that express E. coli PNP (either from lentivirus or MuLv), or when given in combination with vectors that express E. coli PNP.

These results indicate that very little F-Ade is released into the plasma from tumors expressing E. coli PNP after systemic treatment with F-araA. The results with F-dAdo (which releases F-Ade with greater efficiency than F-araA, Table 1) demonstrate that the amount of F-Ade liberated in this setting is both efficacious and well tolerated (Parker, W. B., et al., 2002, Cancer Gene Therapy 9, 1-7).

### Example 25

### Analysis of Normal Tissues Following PNP-Based Therapy

To determine the amount of F-Ade intracellular metabolites, tumors and other tissues will be excised 4 hours after injection of radiolabeled prodrug and the amount of radioactivity determined. A survey of tissues (e.g. liver, lung, kidney, heart, intestine, marrow, brain, spleen, and gonads) will be conducted. There is very little prodrug in plasma 4 hours after injection of F-araAMP (Parker, W. B., et al., 2002, Cancer Gene Therapy 9, 1-7). Therefore the radioactivity associated with tumors should represent metabolites of the prodrug. By comparing tumors that express E. coli PNP with tumors that do not express this enzyme, one can determine the amount of radioactivity associated with the tumor that is due to the cleavage of F-araA or other prodrugs. Illustration of details and accuracy of this technique for monitoring PNP prodrug cleavage in vivo is described in Parker, W. B., et al., 1998, Biochem. Pharmacol. 55, 1673-1681; Parker, W. B., et al., 2002, Cancer Gene Therapy 9, 1-7; Gadi, V. K., et al., 2003, J. Pharm. Exper. Therap. 304, 1280-1284; and Hong, J. S., et al., 2004, Cancer Research 64, 6610-6615.

Organs and tissues may also be analyzed by histopathology following E. coli PNP/F-araAMP at various time points (e.g. 1, 5, 14, 28 days) after therapy. Histopathology and blood analysis (liver and renal function, glucose, and peripheral blood cell counts) may also be monitored. For biochemical analysis, tumors, livers and other tissues are removed from mice, flash frozen on dry ice, and stored at −70° C. until analysis. The samples are mixed with an equal volume of a 10 mM HEPES buffer, pH 7.4 buffer and homogenized in a teflon/glass rotary cell disrupter. The homogenate iscentrifuged at 100,000×g for 60 minutes at 4° C. and then dialyzed against 1000-fold volume of 100 mM HEPES buffer, pH 7.4 containing 20% glycerol. The protein concentration of each sample is determined, and each tissue monitored for PNP activity and APRT as described above.

### Example 26

### Recovery and Detection of Active Virus Following Prodrug Treatment

Levels of replicating adenoviral vector within tumors and other tissues may be monitored at various time points after prodrug treatment (e.g. 1, 3, 5, 7, 14, and 28 days), by harvesting tumors which are then minced/homogenized in a PBS buffer containing EDTA, followed by three freeze-thaw cycles and sonication as described in Demers, G. W., et al., 2003, Cancer Res. 63:4003-8.

The samples are serially diluted and titered on HEK 293 cells by the limiting dilution method (determination of a 50% tissue culture infectious dose (TC ID50) by evaluating cytopathic effect over a ten day period post-infection). All samples are run in triplicate.

A similar assay is used for HSV. In this protocol, tumor tissues are homogenized at 4° C. in DMEM/F12 buffer containing 7% FBS and sterilized milk. Samples are then frozen in liquid nitrogen and incubated at −80° C. for fifteen minutes, followed by three freeze thaw cycles. After a final centrifugation step to remove debris, the supernatant will be titered on Vero cells by standard techniques.

Ectopic viral delivery is evaluated by PCR using virus specific primers and genomic DNA samples extracted from non-tumor tissues. For each PCR reaction, 100 ng of DNA (equivalent to genomic DNA from approximately 1700 cells, 60 pg genomic DNA/cell) is mixed with 50 pmol for each primer pair. Primers specific for HSV tk (thymidine kinase) are (5′-CTTAACAGCGTCAACAGCGT and 5′-CAAAGAGGTGCGGGAGT) described in Parker, J. N., 2006, Vaccine, 24(10):1644-52, are used. Primers specific for adenovirus are derived from the hexon coding region: 5′-ACTATATGGACAACGTCAACCCATT-3′ (forward) and 5′-AACTTCTGAGGCACCTGGATGT-3′ (reverse). Control PCRs include no template (negative control) and positive controls in which control tissue DNA samples are “spiked” with purified viral DNA to evaluate possible PCR inhibitors. As alternatives, in situ hybridization protocols as described in Gadi, V. K., et al., 2000, Gene Therapy, 7:1738-1743 are used and along with adapted commercially available, biotinylated virus-specific DNA probes (Enzo Diagnostics), and/or immunohistochemistry using viral antigen specific antibodies (e.g. against adenoviral hexon protein (Chemicon) or rabbit anti-HSV (Dako Corp)). Primers for detecting APRT mRNA expressed from adenovirus or HSV may also be used.

### Example 27

### Studies to Compare In Vivo F-AraAMP Treatment Regimens

Treatment-schedule-dependency studies are undertaken early in the course of in vivo evaluation of drugs. It is necessary to identify the optimal treatment schedule in order to design subsequent comparative studies. The schedules most often used in schedule-dependency trials of prodrugs such as F-araAMP typically include: a single bolus dose, once daily for five or nine doses, once every 4 days for three or four doses, and some version of every 3 hours for three or eight doses for multiple courses at 4-day intervals. A range of dosage levels is used for each schedule to provide dose-response data and to include a toxic dosage level for a benchmark, since it is important to compare various treatment schedules at equitoxic dosages. The antitumor activity observed with a given compound may exhibit a striking dependence on the drug treatment schedule employed. For agents such as F-Ade, strong antitumor activity has been observed after relatively short F-araAMP treatment schedules (e.g., Q1D×3 days; or Q1D×3 days, q4h×3). Examples of modifications of F-araAMP schedules for use with E. coli PNP include 160 mg/kg q1d×3-q4h×3; 100 mg/kg q2h×5; qld×3; 167 mg/kg q1d×3-q4h×3; 250 mg/kg q1d×3; and 25-100 mg/kg q2h×5, q1d×3.

### Example 28

### Administration Optimization

Regarding methods including administration of replicating viruses encoding PNP and APRT, replicating vaccinia, replicating retrovirus and HSV, bystander killing may be optimized by applying vector at a time when 1) tumors are no longer responding adequately to viral oncolysis alone, and 2) E. coli PNP activity is sufficient to mediate strong tumor regressions and tumor-free survivors with fludarabine. This time point allows a two pronged attack against both dividing tumor cells using oncolytic virus and quiescent, bystander cells using E. coli PNP, APRT and prodrug to effectively destroy the tumor mass. In addition, such an approach allows for elimination of replicating vector, an effect that could be desirable from the standpoint of long-term safety.

Various regulatory elements may be incorporated into PNP and/or APRT expression vectors, such as lentivirus herpes simplex virus and adenovirus. A CMV promoter allows for strong expression of the transgenes. In addition, the EF-1α promoter available from Invitrogen, the WPRE regulatory element described in relation to lentiviral constructs and/or hypoxia responsive elements (HREs) may be included.

### Example 29

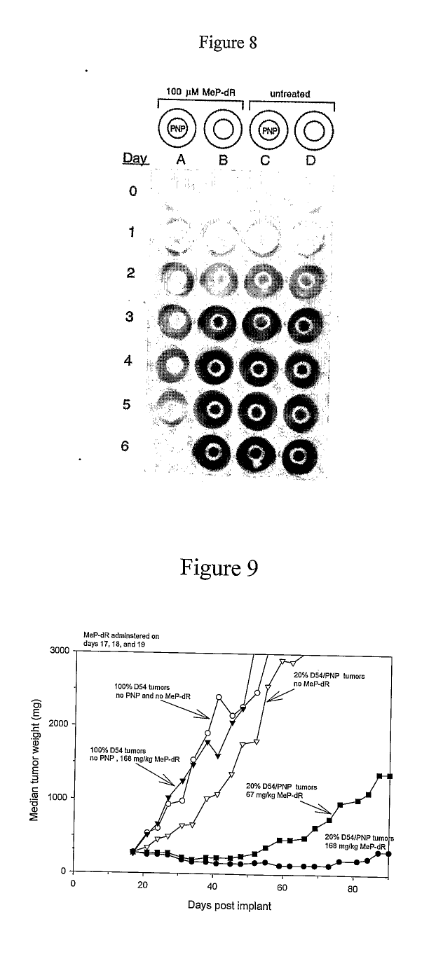

Gap junctions and cell-to-cell contact are not required using methods and compositions according to the present invention. Bystander activity of MeP-dR in D54 human glioma cells expressing E. coli PNP is illustrated in vitro. D54 cells seeded inside or outside a cloning ring (removed) are separated by thin barrier (uncrossable) of vacuum grease. All surrounding cells (outside the ring) are D54 parental cells having no E. coli PNP expression. In columns A and C shown in FIG. 8, cells inside the ring are D54-PNP cells, and in columns B and D, the inside cells are D54 parental cells. Columns A and B are treated continuously with 100 micromolar MeP-dR for 6 days. On each day, a row of cells was fixed and stained with crystal violet to monitor cell growth.

Because both the PNP prodrug and purine base are freely membrane permeant, gap junctions or cell-to-cell contact are not required for bystander killing mediated by E. coli PNP. An example of this is shown in FIG. 8 and related in vitro studies pertaining to PNP as described in Hughes, B. W., et al., 1998, J. Biol. Chem. 273, 2322-2328.

### Example 30

### Bystander Killing In Vivo Following Lentiviral Transduction

Bystander killing by MeP-dR when E. coli PNP is expressed using MuLV is shown in FIG. 9. Twenty percent PNP-expressing (D54-PNP, MuLv transduced) and eighty percent non-expressing (D54, parental) cells are used to establish tumors (PNP activity 79±39 units in tumors harvested on day 17, where 1 PNP unit cleaves 1 nmole MeP-dR per milligram tumor tissue per hour). Results in parental tumors (zero PNP activity on day 17) with or without MeP-dR are also shown. These treatments are well tolerated without limiting weight loss or lethality. At least six animals are studied per group (median tumor weights are shown). PNP expression in MuLv was driven by an SV40 promoter. Mice are given non-absorbable antibiotics in their drinking water prior to treatment with high doses of MeP-dR (e.g. 168 mg/kg/d×3d) to minimize intestinal cleavage of the compound (Bharara, S., et al., 2005, Human Gene Therapy 16:339-347.). Antibiotics are not co:administered for lower doses of MeP-dR, or with less avid substrates such as fludarabine. MeP-dR treatment groups in PNP expressing tumors are significantly different from non-treatment groups (p<0.002).

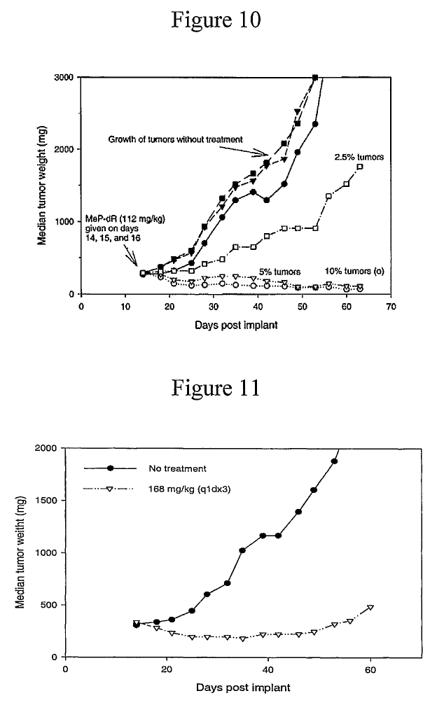

In order to confirm activity of MeP-dR as an E. coli PNP substrate by an independent method of expression, a more active vectoring system is used including cloning and purifying recombinant lentivirus, expression of E. coli PNP in D54 tumor cells, isolating and propagating new D54-PNP tumor cell lines, monitoring tumor growth in animals. D54 glioma tumors are established from a stable cell line expressing E. coli PNP by a strong, CMV-based promoter in a lentiviral vector. FIG. 10 illustrates results. Open Circles: Tumors established from 10% PNP expressing/90% non-expressing tumor cells; PNP activity 9600±1300 units on day 14. Open Triangles: 5% expressing, 95% non-expressing tumor cells; PNP activity 5500±1700 units. Open Squares: Tumors established from 2.5% expressing, 97.5% non-expressing (PNP activity 3600±530 units). Closed symbols depict growth of corresponding (10%, 5%, 2.5%) PNP tumors treated with vehicle (saline control). At least six animals are studied per group. Identically treated animals from each study arm are sacrificed just prior to initiation of MeP-dR therapy (day 14) to measure intratumoral PNP acitivity. Antitumor effects in MeP-dR treatment groups are significantly different from non-treatment groups (p<0.05, 2.5% tumors; and p<0.005 for 5% and 10% tumors). These experiments test the influence of substantially higher PNP activities on bystander killing in vivo. PNP activity in 300 mg tumors comprised of 100% lentivirus transduced cells is found to be approximately 126,000 PNP units (one unit=1 nmole MeP-dR converted per mg tumor tissue per hour). When the level of E. coli PNP is studied using lentiviral expression, improvements in bystander killing are observed as shown in FIG. 10.

Similar experiments are performed with co-expressed E. coli PNP and human APRT in order to observe the improved effects of including APRT expression vectors to overexpress APRT.

### Example 31

D54 tumors are established as in the previous example except that 1% of the cells used to establish the tumors express E. coli PNP (99% parental, 1% non-PNP expressing cells) (p=0.001). PNP activity is 1500 and 1740 units in two control tumors on the day of MeP-dR treatment (day 17).

Increasing the level of PNP activity allows bystander killing in vivo with MeP-dR when a fraction (2.5-10%) of tumor cells express the gene. Studies of lower percentages of E. coli PNP expression in vivo using higher doses of MeP-dR are also conducted. A result with 1% PNP expressing cells is shown in FIG. 11.

The half life of MeP-dR in serum is short (on the order of 15-20 minutes) (Gadi, V. K., et al., 2003, J. Pharm. Exper. Therap. 304, 1280-1284), and the doubling time of the tumors shown here is 10-15 days. The results therefore indicate that both dividing and non-dividing tumor cells are being killed over a relatively short course of prodrug (3 doses MeP-dR). The findings also indicate that increasing the amount of intratumoral enzyme, MeP-dR or the amount of conversion of MeP-dR to cytotoxin can safely augment bystander killing.

Similar experiments are performed with addition of human APRT expression constructs in these cells to produce improved bystander killing.

### Example 32

Tumor responses to administration of E. coli PNP and/or human APRT expression constructs and fludarabine.

F-araA is less active than MeP-dR as a substrate for E. coli PNP based upon Vmax/km of the compounds, as calculated from Table 1. However, F-araAMP is capable of mediating anti-tumor effects in vivo. As noted above, tumors established using a first generation system (MuLv-PNP transduced cells, PNP under regulatory control of SV40 promoter) exhibit regressions following MeP-dR (Gadi, V. K., et al., 2003, J. Pharm. Exper. Therap. 304, 1280-1284; and FIG. 9). F-araAMP in these same tumors confers regressions lasting ˜30 days when 100% of cells expressed E. coli PNP, but tumors subsequently escapefrom F-araAMP therapy and progress. Moreover, little or no bystander killing is observed in vivo with F-araAMP following E. coli PNP transduction with MuLv.

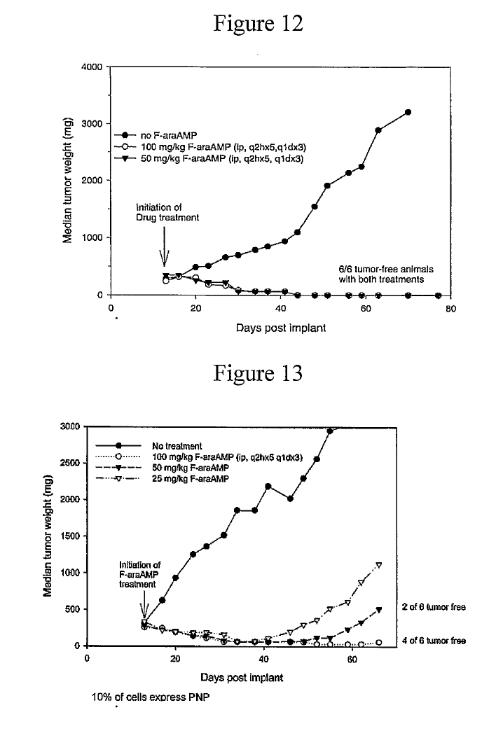

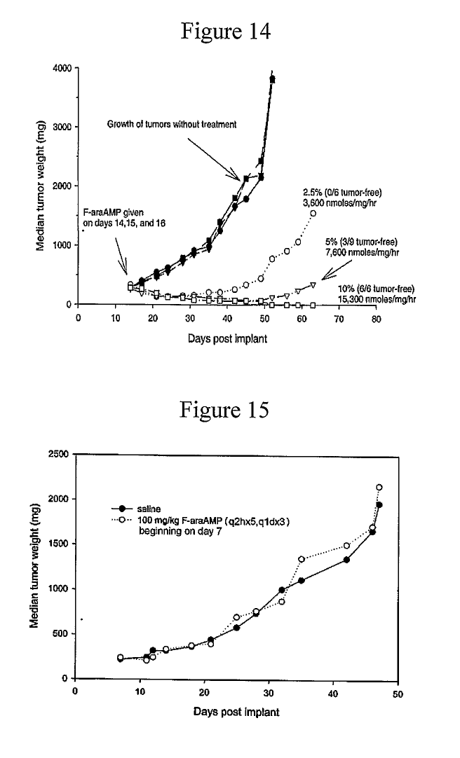

FIG. 12 depicts a study in which 100% of cells express E. coli PNP using lentivirus. Complete regression and cures of tumors (12 of 12) are obtained with F-araAMP following a 3 day treatment schedule. Tumors with lower proportions of PNP-expressing cells (10%, 5%, 2.5%), exhibit dose dependence upon both the amount of prodrug added (FIG. 13) and the intratumoral PNP activity (FIG. 14) compared to controls (FIG. 15). Anti-tumor effects are observed with F-araAMP when as few as 2.5% of tumor cells in vivo express PNP. Adjusting the schedule of F-araAMP may also improve bystander killing in vivo (FIG. 16).

In summary, D54 tumors are a stringent in vivo model for preclinical testing since they are slow growing in mice and resistant to standard cancer agents used against CNS tumors. For example, BCNU, a clinically approved anti-glioma chemotherapy, has minimal activity against these tumors at maximally tolerated doses. The findings provided indicate the ability to treat otherwise refractory tumors using methods and compositions according to the present invention. Although F-araA is less active as a PNP substrate than MeP-dR, it liberates a more potent agent (F-Ade vs MeP). The circulating half life of F-araA is longer than MeP-dR (50 minutes vs. appx. 20 minutes) and peak levels are significantly higher with F-araA than MeP-dR (Parker, W. B., et al., 2002, Cancer Gene Therapy 9, 1-7.).

Similar experiments are performed with co-expressed E. coli PNP and human APRT in order to observe the improved effects of including APRT expression vectors to overexpress APRT.

FIG. 12 shows cures of mice bearing lentiviral transduced D54 tumors expressing E. coli PNP and treated with F-araAMP. Tumors in which 100% of cells expressed E. coli PNP are treated with a maximally tolerated dose (MTD, 100 mg/kg schedule, open circles) or half the MTD (closed triangles). Six animals are included per treatment group. All animals in both F-araAMP treated groups are cured of their tumors. Tumor regressions are observed in these studies without excessive weight loss or other undesired sequelae. PNP activity in these tumors is 126,000±16,000 units on the day of drug therapy. F-araAMP treatment groups are significantly different from non-treatment group (p<0.0001).

FIG. 13 shows that the anti-tumor effect of F-araAMP exhibits dose dependence on the level of prodrug administered. Tumors are established from an inoculum in which 10% of cells expressed E. coli PNP. PNP expression on the day of drug treatment is 14,200±681 PNP units. Anti-tumor efficacy is greater at 100 mg/kg F-araAMP (open circles) than at 50 mg/kg (closed triangles) or 25 mg/kg (open triangles) given over a standard 3 day schedule (q2h×5, q1d×3 days). F-araAMP treatment groups are significantly different from non-treatment group (p<0.001).

FIG. 14 shows that tumor regression using E. coli PNP and F-araAMP also exhibits dose dependence on the level of suicide gene expression. D54 glioma tumors are established with decreasing proportions of PNP expressing cells (and increasing non-expressing (parental) cells). (open symbols: F-araAMP therapy; closed symbols: no prodrug therapy; circles: 2.5%; triangles 5%, and squares 10% PNP transduced cells). F-araAMP was 100 mg/kg q2h×5, q1d×3. F-araAMP treatment groups are significantly different from non-treatment groups (p<0.001).

FIG. 15 illustrates that F-araAMP has no effect on control (no PNP expression) D54 tumors. Administration of F-araAMP (open circles, by the standard of 3 day schedule) using a control transgene (green fluorescent protein, expressed from an otherwise identical vector to Lenti-PNP) reveals no anti-tumor effects (no PNP expression).

FIG. 16 shows that multiple F-araAMP schedules exhibit bystander killing. Tumors comprised of 5% PNP expressing cells and 2 different fludarabine schedules are shown. F-araAMP treatment groups are significantly different from non-treatment group (p<0.001).

### Example 33

### Delivery of E. coli PNP and/or APRT to Tumor Cells Using an Attenuated Adenoviral Vector

Ad-PNP mediated cell killing in vitro is shown in FIG. 20. Confluent HeLa cells are infected with Ad-PNP at various MOIs. MeP-dR is added at 40 micrograms/milliliter in one set of cells at 24 hours post infection. At 6 days post infection, all cultures are stained with Crystal Violet to visualize living cells.

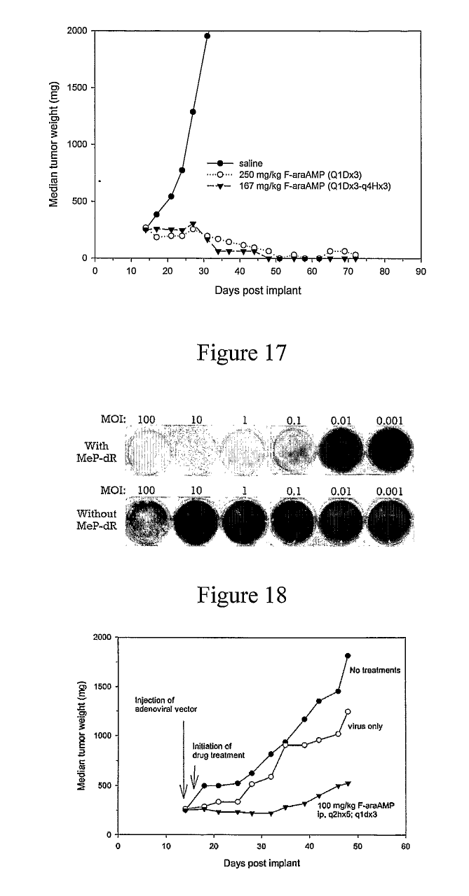

FIG. 17 depicts results using an Ela deleted adenoviral vector encoding E. coli PNP as described herein. Adenoviral MOI's of 0.1-100 in vitro are sufficient to eliminate populations of cancer cells in combination with MeP-dR by this assay.

Similar experiments are performed with co-expressed E. coli PNP and human APRT in order to observe the improved effects of including APRT expression vectors to overexpress APRT.

### Example 34

### Anti-tumor effects of F-araAMP following delivery of E. coli PNP by Ad-PNP are shown in FIG. 18. D54 human glioma tumors (appx. 250 mg) are injected with an Ad-PNP (2×10⁹ PFU, open circles) or saline (closed circles). PNP activity in tumor extracts taken two days after vector administration is approximately 5000 PNP units. Ad-PNP together with F-araAMP confer slowing of tumor growth (closed triangles) (p<0.002 compared to virus alone or no treatment controls). No limiting weight loss, animal deaths or other toxicities were noted in this study.

Localization studies in D54 tumors in mice following direct inoculation of Ela deleted adenoviral vector encoding E. coli PNP as described herein indicate compartmentalized transgene expression presumptively along the needle tracks used to administer the vector. The results shown in FIG. 21 indicate that tumor growth slows due to E. coli PNP expression in solid tumors followed by F-araAMP dosing.

Similar experiments are performed with co-expressed E. coli PNP and human APRT in order to observe the improved effects of including APRT expression vectors to overexpress APRT.

### Protein and Nucleic Acid Sequences Referred to Herein:

| SEQ ID No. 1 | |
|---|---|
| Homo sapiens adenine phosphoribosyltransferase (APRT) | |
| Protein: | |
| MADSELQLVEQRIRSFPDFPTPGVVFRDISPVLKDPASFRAAIGLLARHLKATHGG | |
| RIDYIAGLDSRGFLFGPSLAQELGLGCVLIRKRGKLPGPTLWASYSLEYGKAELEI | |
| QKDALEPGQRVVVVDDLLATGGTMNAACELLGRLQAEVLECVSLVELTSLKGR | |
| EKLAPVPFFSLLQYE | |
| SEQ ID No. 2 | |
| Homo sapiens adenine phosphoribosyltransferase (APRT) | |
| DNA: coding sequence 36-578 | |
| 1 | gcgctcgggc tgccgctggc tcttcgcacg cggccatggc cgactccgag ctgcagctgg |
| 61 | ttgagcagcg gatccgcagc ttccccgact tccccacccc aggcgtggta ttcagggaca |
| 121 | tctcgcccgt cctgaaggac cccgcctcct tccgcgccgc catcggcctc ctggcgcgac |
| 181 | acctgaaggc gacccacggg ggccgcatcg actacatcgc aggcctagac tcccgaggct |
| 241 | tcctctttgg cccctccctg gcccaggagc ttggactggg ctgcgtgctc atccgaaagc |
| 301 | gggggaagct gccaggcccc actctgtggg cctcctattc cctggagtac gggaaggctg |
| 361 | agctggagat tcagaaagac gccctggagc caggacagag ggtggtcgtc gtggatgatc |
| 421 | tgctggccac tggtggaacc atgaacgctg cctgtgagct gctgggccgc ctgcaggctg |
| 481 | aggtcctgga gtgcgtgagc ctggtggagc tgacctcgct taagggcagg gagaagctgg |
| 541 | cacctgtacc cttcttctct ctcctgcagt atgagtgacc acagggcctc ccagcccaac |
| 601 | atctccagct ggatcccagg gaaatatcag ccttgggcaa ctgcagtgac caggggcacc |
| 661 | ggctgcccac agggaacaca ttcctttgct ggggttcagc gcctctcctg gggctggaag |
| 721 | tgccaaagcc tggggcaaag ctgtgtttca gccacactga acccaattac acacagcggg |
| 781 | agaacgcagt aaacagcttt cccacaa |

| SEQ ID No. 3 | |
|---|---|
| E. coli PNP | |
| Protein: | |
| Met Ala Thr Pro His Ile Asn Ala Glu Met Gly Asp Phe Ala Asp Val | |
| Val Leu Met Pro Gly Asp Pro Leu Arg Ala Lys Tyr Ile Ala Glu Thr | |
| Phe Leu Glu Asp Ala Arg Glu Val Asn Asn Val Arg Gly Met Leu Gly | |
| Phe Thr Gly Thr Tyr Lys Gly Arg Lys Ile Ser Val Met Gly His Gly | |
| Met Gly Ile Pro Ser Cys Ser Ile Tyr Thr Lys Glu Leu Ile Thr Asp | |
| Phe Gly Val Lys Lys Ile Ile Arg Val Gly Ser Cys Gly Ala Val Leu | |
| Pro His Val Lys Leu Arg Asp Val Val Ile Gly Met Gly Ala Cys Thr | |
| Asp Ser Lys Val Asn Arg Ile Arg Phe Lys Asp His Asp Phe Ala Ala | |
| Ile Ala Asp Phe Asp Met Val Arg Asn Ala Val Asp Ala Ala Lys Ala | |
| Leu Gly Ile Asp Ala Arg Val Gly Asn Leu Phe Ser Ala Asp Leu Phe | |
| Tyr Ser Pro Asp Gly Glu Met Phe Asp Val Met Glu Lys Tyr Gly Ile | |
| Leu Gly Val Glu Met Glu Ala Ala Gly Ile Tyr Gly Val Ala Ala Glu | |
| Phe Gly Ala Lys Ala Leu Thr Ile Cys Thr Val Ser Asp His Ile Arg | |
| Thr His Glu Gln Thr Thr Ala Ala Glu Arg Gln Thr Thr Phe Asn Asp | |
| Met Ile Lys Ile Ala Leu Glu Ser Val Leu Leu Gly Asp Lys Glu | |
| SEQ ID No. 4 | |
| E. coli PNP | |
| DNA: | |
| atggctaccc cacacattaa tgcagaaatg ggcgatttcg ctgacgtagt tttgatgcca | 60 |
| ggcgacccgc tgcgtgcgaa gtatattgct gaaactttcc ttgaagatgc ccgtgaagtg | 120 |
| aacaacgttc gcggtatgct gggcttcacc ggtacttaca aaggccgcaa aatttccgta | 180 |
| atgggtcacg gtatgggtat cccgtcctgc tccatctaca ccaaagaact gatcaccgat | 240 |
| ttcggcgtga agaaaattat ccgcgtgggt tcctgtggcg cagttctgcc gcacgtaaaa | 300 |
| ctgcgcgacg tcgttatcgg tatgggtgcc tgcaccgatt ccaaagttaa ccgcatccgt | 360 |
| tttaaagacc atgactttgc cgctatcgct gacttcgaca tggtgcgtaa cgcagtagat | 420 |
| gcagctaaag cactgggtat tgatgctcgc gtgggtaacc tgttctccgc tgacctgttc | 480 |
| tactctccgg acggcgaaat gttcgacgtg atggaaaaat acggcattct cggcgtggaa | 540 |
| atggaagcgg ctggtatcta cggcgtcgct gcagaatttg gcgcgaaagc cctgaccatc | 600 |
| tgcaccgtat ctgaccacat ccgcactcac gagcagacca ctgccgctga gcgtcagact | 660 |
| accttcaacg acatgatcaa aatcgcactg gaatccgttc tgctgggcga taaagagtaa | 720 |

Any patents or publications mentioned in this specification are incorporated herein by reference to the same extent as if each individual publication is specifically and individually indicated to be incorporated by reference. In particular, U.S. Provisional Patent Application Ser. No. 60/681,305, filed May 16, 2005, is hereby incorporated by reference in its entirety.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

We claim:

1. A method of inhibiting a mammalian cell, comprising:
introducing an expression vector comprising a nucleotide sequence encoding an adenine phosphoribosyltransferase into the mammalian cell; and
contacting the mammalian cell with an effective amount of a substrate for the adenine phosphoribosyltransferase, wherein activation of the substrate for the adenine phosphoribosyltransferase by the adenine phosphoribosyltransferase yields a compound toxic to the mammalian cell, thereby inhibiting the mammalian cell.

2. The method of claim 1 wherein the nucleotide sequence encoding an adenine phosphoribosyltransferase encodes a mammalian adenine phosphoribosyltransferase.

3. The method of claim 1 wherein the nucleotide sequence encoding an adenine phosphoribosyltransferase encodes a human adenine phosphoribosyltransferase.

4. The method of claim 1 wherein the nucleotide sequence encoding an adenine phosphoribosyltransferase encodes a human adenine phosphoribosyltransferase comprising amino acids 1-180 of Seq ID No 1.

5. The method of claim 1 wherein the substrate for the adenine phosphoribosyltransferase is a purine analog.

6. The method of claim 1 wherein the purine analog is selected from the group consisting of: 6-methylpurine, 2-fluoroadenine and a combination thereof.

7. The method of claim 1 wherein the vector is selected from the group consisting of: a plasmid and a virus.

8. The method of claim 7 wherein the virus is selected from the group consisting of: an adenovirus, a herpes virus, an adeno-associated virus and a lentivirus.

9-11. (canceled)

12. A method of inhibiting a mammalian cell, comprising:
introducing a first expression vector comprising a nucleotide sequence encoding a prokaryotic purine nucleoside phosphorylase into a mammalian cell;
introducing a second expression vector comprising a nucleotide sequence encoding an adenine phosphoribosyltransferase into the mammalian cell; and
contacting the mammalian cell with an effective amount of a prodrug which is a substrate for the purine nucleoside phosphorylase, wherein cleavage of the prodrug by the purine nucleoside phosphorylase yields a substrate for the adenine phosphoribosyltransferase, and wherein activation of the substrate for the adenine phosphoribosyltransferase by the adenine phosphoribosyltransferase yields a compound toxic to the mammalian cell, thereby inhibiting the mammalian cell.

13. The method of claim 12 wherein the purine nucleoside phosphorylase is an E. coli purine nucleoside phosphorylase or T. vaginalis purine nucleoside phosphorylase.

14. The method of claim 12 wherein the first expression vector comprising the nucleotide sequence encoding a prokaryotic purine nucleoside phosphorylase and the second expression vector comprising the nucleotide sequence encoding an adenine phosphoribosyltransferase are each independently selected from the group consisting of: a plasmid and a virus.

15. The method of claim 12 wherein the first expression vector and the second expression vector are the same, the prokaryotic purine nucleoside phosphorylase and the adenine phosphoribosyltransferase encoded by a bicistronic nucleic acid sequence.

16. The method of claim 12 wherein the nucleotide sequence encoding an adenine phosphoribosyltransferase encodes a mammalian adenine phosphoribosyltransferase.

17. The method of claim 12 wherein the substrate for the purine nucleoside phosphorylase is a purine nucleoside analog.

18. (canceled)

19. The method of claim 12 wherein the substrate for the adenine phosphoribosyltransferase is a purine analog.

20. A composition, comprising:
a bicistronic expression construct comprising a first nucleic acid encoding a prokaryotic purine nucleoside phosphorylase and a second nucleic acid encoding a mammalian adenine phosphoribosyltransferase, the first and second nucleic acids both operably linked to a promoter.

21. The composition of claim 20 wherein the bicistronic expression construct further comprises an internal ribosome entry site disposed between the first nucleic acid encoding a prokaryotic purine nucleoside phosphorylase and the second nucleic acid encoding a mammalian adenine phosphoribosyltransferase.

22. The composition of claim 20 wherein the first nucleic acid encoding a prokaryotic purine nucleoside phosphorylase encodes an E. coli purine nucleoside phosphorylase.

23. The composition of claim 22 wherein the first nucleic acid encoding an E. coli purine nucleoside phosphorylase encodes a protein which is at least 90% identical to a E. coli purine nucleoside phosphorylase of SEQ ID No. 3.

24. The composition of claim 22 wherein the first nucleic acid encoding a prokaryotic purine nucleoside phosphorylase is at least 80% identical to a E. coli purine nucleoside phosphorylase encoding portion of a nucleic acid of SEQ ID No. 4.

25. The composition of claim 20 wherein the second nucleic acid encoding a mammalian adenine phosphoribosyltransferase encodes a human adenine phosphoribosyltransferase.

26. The composition of claim 25 wherein the second nucleic acid encoding a human adenine phosphoribosyltransferase encodes a protein which is at least 90% identical to a human adenine phosphoribosyltransferase of SEQ ID No. 1.

27. The composition of claim 25 wherein the second nucleic acid encoding a human adenine phosphoribosyltransferase is at least 80% identical to a human adenine phosphoribosyltransferase encoding portion of a nucleic acid of SEQ ID No. 2.

28-29. (canceled)

30. An expression vector comprising:
a nucleic acid sequence encoding., a human adenine phosphoribosyltransferase nucleic acid encoding a human adenine phosphoribosyltransferase is at least 80% identical to a human adenine phosphoribosyltransferase encoding portion of a nucleic acid of SEQ ID No. 2.

31. The expression vector of claim 30 wherein the nucleic acid encoding a human adenine phosphoribosyltransferase encodes a protein which is at least 90% identical to a human adenine phosphoribosyltransferase of SEQ ID No. 1.

32. A pharmaceutical composition for inhibiting a cell, comprising:
an expression vector of claim 30; and
a pharmaceutically acceptable carrier.

33. The pharmaceutical composition of claim 32, further comprising:
an expression vector comprising a nucleotide sequence encoding a prokaryotic purine nucleoside phosphorylase.

34. The pharmaceutical composition of claim 33 wherein the expression vector comprising a nucleotide sequence encoding an adenine phosphoribosyltransferase and the expression vector comprising a nucleotide sequence encoding a prokaryotic purine nucleoside phosphorylase and the same vector, the nucleotide sequence encoding an adenine phosphoribosyltransferase and the nucleotide sequence encoding a prokaryotic purine nucleoside phosphorylase operably connected to a regulatory element in a bicistronic nucleic acid.

35. (canceled)


SEQUENCE LISTING (4 sequences)

SEQ ID NO: 1 - protein, 180 aa, Homo sapiens
MADSELQLVEQRIRSFPDFPTPGVVFRDISPVLKDPASFRAAIGLLARHLKATHGGRIDYIAGLDSRGFLFGPSLAQELGLGCVLIRKRGKLPGPTLWASYSLEYGKAELEIQKDALEPGQRVVVVDDLLATGGTMNAACELLGRLQAEVLECVSLVELTSLKGREKLAPVPFFSLLQYE

SEQ ID NO: 2 - DNA, 807 bp, Homo sapiens
gcgctcgggctgccgctggctcttcgcacgcggccatggccgactccgagctgcagctggttgagcagcggatccgcagcttccccgacttccccaccccaggcgtggtattcagggacatctcgcccgtcctgaaggaccccgcctccttccgcgccgccatcggcctcctggcgcgacacctgaaggcgacccacgggggccgcatcgactacatcgcaggcctagactcccgaggcttcctctttggcccctccctggcccaggagcttggactgggctgcgtgctcatccgaaagcgggggaagctgccaggccccactctgtgggcctcctattccctggagtacgggaaggctgagctggagattcagaaagacgccctggagccaggacagagggtggtcgtcgtggatgatctgctggccactggtggaaccatgaacgctgcctgtgagctgctgggccgcctgcaggctgaggtcctggagtgcgtgagcctggtggagctgacctcgcttaagggcagggagaagctggcacctgtacccttcttctctctcctgcagtatgagtgaccacagggcctcccagcccaacatctccagctggatcccagggaaatatcagccttgggcaactgcagtgaccaggggcaccggctgcccacagggaacacattcctttgctggggttcagcgcctctcctggggctggaagtgccaaagcctggggcaaagctgtgtttcagccacactgaacccaattacacacagcgggagaacgcagtaaacagctttcccacaa

SEQ ID NO: 3 - protein, 239 aa, E. coli
MATPHINAEMGDFADVVLMPGDPLRAKYIAETFLEDAREVNNVRGMLGFTGTYKGRKISVMGHGMGIPSCSIYTKELITDFGVKKIIRVGSCGAVLPHVKLRDVVIGMGACTDSKVNRIRFKDHDFAAIADFDMVRNAVDAAKALGIDARVGNLFSADLFYSPDGEMFDVMEKYGILGVEMEAAGIYGVAAEFGAKALTICTVSDHIRTHEQTTAAERQTTFNDMIKIALESVLLGDKE

SEQ ID NO: 4 - DNA, 720 bp, E. coli
atggctaccccacacattaatgcagaaatgggcgatttcgctgacgtagttttgatgccaggcgacccgctgcgtgcgaagtatattgctgaaactttccttgaagatgcccgtgaagtgaacaacgttcgcggtatgctgggcttcaccggtacttacaaaggccgcaaaatttccgtaatgggtcacggtatgggtatcccgtcctgctccatctacaccaaagaactgatcaccgatttcggcgtgaagaaaattatccgcgtgggttcctgtggcgcagttctgccgcacgtaaaactgcgcgacgtcgttatcggtatgggtgcctgcaccgattccaaagttaaccgcatccgttttaaagaccatgactttgccgctatcgctgacttcgacatggtgcgtaacgcagtagatgcagctaaagcactgggtattgatgctcgcgtgggtaacctgttctccgctgacctgttctactctccggacggcgaaatgttcgacgtgatggaaaaatacggcattctcggcgtggaaatggaagcggctggtatctacggcgtcgctgcagaatttggcgcgaaagccctgaccatctgcaccgtatctgaccacatccgcactcacgagcagaccactgccgctgagcgtcagactaccttcaacgacatgatcaaaatcgcactggaatccgttctgctgggcgataaagagtaa